(19) United States
(12) Patent Application Publication
(10) Pub. No.: US 2015/0306167 A1
(43) Pub. Date: Oct. 29, 2015

(54) ALPINIA SPP. EXTRACTS FOR TREATING IRRITABLE BOWEL SYNDROME

(71) Applicant: MEDICAL AND PHARMACEUTICAL INDUSTRY TECHNOLOGY AND DEVELOPMENT CENTER, New Taipei City (TW)

(72) Inventors: Jui-Ching CHEN, New Taipei City (TW); Che-Yi LIN, Tainan City (TW); Chih-Sheng HUNG, New Taipei City (TW); Pei-Fan WU, New Taipei City (TW); Fen CHEN, Taipei City (TW)

(73) Assignee: Medical and Pharmaceutical Industry Technology and Development Center, New Taipei City (TW)

(21) Appl. No.: 14/650,745

(22) PCT Filed: Dec. 30, 2013
(86) PCT No.: PCT/CN2013/090950

§ 371 Date: Jun. 9, 2015

Related U.S. Application Data
(60) Provisional application No. 61/747,216, filed on Dec. 29, 2012.

(51) Int. Cl. A61K 36/9062; A61K 31/352; A61K 31/122

(52) U.S. Cl. CPC A61K 36/9062; A61K 31/122; A61K 31/352

(57) ABSTRACT

Disclosed herein is the use of an extract from Alpinia spp. for the preparation of a medicament for the treatment of irritable bowel syndrome (IBS). The extract is prepared by extracting fresh and/or dried fruits of Alpinia spp. plant with a solvent such as a supercritical fluid, water, 10-95% (v/v) ethanol, acetone, ethyl acetate or n-hexane, to obtain an extract suitable for the preparation of IBS medicaments. In some embodiments, active compounds with anti-abnormal defecation activity and/or analgesia effect are isolated from the water extract of Alpinia spp.; these compounds are therefore potential lead compounds for use as medicaments for treating IBS.

### BACKGROUND OF THE INVENTION

1. Field of the Invention

The present disclosure relates to the treatment of irritable bowel syndrome (IBS). More particularly, the disclosed invention relates to the use of an extract obtaining from Alpinia spp. as an agent in the treatment of IBS.

2. Description of Related Art

Irritable bowel syndrome (IBS) is a common functional gastrointestinal disorder that is not well-linked to any readily measured physiological abnormality. Symptoms of IBS are a product of quantitative differences in the motor reactivity of the intestinal tract, and increased sensitivity to stimuli or spontaneous contractions. Hence, IBS also goes by the name of spastic bowl syndrome, mucous bowl syndrome, or nervous bowl syndrome; however, one shall not confuse IBS with spastic colitis or ulcerative colitis (e.g., Crohn's disease). Despite advance in our understanding of basic neuroenterological mechanisms and the role of efforts and transmitters in the brain-gut axis, a reliable biologic marker of IBS has yet to be identified.

According to the guidance for industry published by US Food and Drug Administration (FDA) entitled “Irritable Bowel Syndrome-Clinical Evaluation of Drugs for Treatment”, the two major IBS signs and symptoms are abnormal defecation and abdominal pain. Hence, only when a compound or a composition exhibits the capability of ameliorating or alleviating both the IBS symptoms, i.e., abnormal defecation, and abdominal pain and/or abdominal discomfort, then such compound or composition may be qualified as a potential IBS medicament.

Therapeutically medicament commonly used for the treatment of IBS includes 5-hydroxytryptamine 3 (5-HT₃) receptor antagonist (e.g., ondansetron, alosetron, cilansetron and/or granisetron), 5-hydroxytryptamine 4 (5-HT₄) receptor agonist (e.g., tegaserod), antispasmodic agents, muscle relaxants and tricyclic antidepressants. Among them, alosetron is reported to improve clinical symptoms in about 50% IBS patients, however, it is also said to result in side effects such as constipation and ischemia colitis among 30-35% IBS patients. As to the 5-HT₄ receptor agonist (i.e., tegaserod), it offers limited therapeutic effects on IBS, yet the risk of IBS patients in developing cardiovascular diseases increases significantly, and is subsequently retracted from the market in the year of 2007 by the order of FDA.

In view of the above, there remains a continue interest in identifying new methods and/or agents for treating IBS.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure is based, at least in part, on the discovery that the extract form Alpinia spp. plant may act as an effective agent in the treatment of a subject diagnosed or suspected of having irritable bowel syndrome (IBS). Therefore, one aspect of the present invention pertains to an extract of Alpinia spp. (hereinafter, the Alpinia spp. extract) for manufacturing a medicament for treating IBS in a subject, in which the medicament is effective in ameliorating the symptoms of abnormal defecation and abdominal pain associated with IBS.

According to embodiments of the present invention, Alpinia spp. may be any of Alpinia oxyphylla, Alpinia zerumbet, Alpinia hainanensis or Alpinia galanga. The Alpinia spp. extract is prepared from a component of the plant of Alpinia spp., specifically, the fresh or dried fruits of Alpinia spp.

According to embodiments of the present invention, the Alpinia spp. extract is prepared by a method that includes steps of,

(a) extracting Alpinia spp. with a first solvent selected from the group consisting of a supercritical fluid (SFC), water, C₁₋₄ alcohol, acetone, ethyl acetate, and n-hexane; and

(b) drying the extract of step (a).

According to some embodiments of the present disclosure, the first solvent is SFC, which is selected from the group consisting of carbon dioxide, water, methane, ethane, propane, ethylene, propylene, methanol, ethanol and acetone. In one example, the SFC is liquid carbon dioxide. According to other embodiments of the present disclosure, the method may further comprise a co-solvent of the SFC. The co-solvent may be methanol or ethanol.

According to optional embodiments of the present disclosure, the method may further include step of, (a-1) adding a second solvent that is any of water, C₁₋₄ alcohol, acetone, ethyl acetate, or n-hexane to the extract before proceeding to the step (b). In one example, the first solvent is water, and the second solvent is 95% (v %) ethanol.

In some embodiments, the method may further include step of, (a-2) subjecting the extract of the step (a-1) to column chromatography before proceeding to the step (b).

In other embodiments, the method may further include the steps of, (c) dissolving the dried product of step (b) in a second solvent that is any of water, C₁₋₄ alcohol, acetone, ethyl acetate, or n-hexane; and (d) concentrating or drying the product of step (c).

In the example in which the first solvent is 50% (v %) ethanol and the second solvent is 95% (v %) ethanol, the method further includes the step of, (e) subjecting the solution of step (c) to column chromatography by eluting the column in sequence with 20% (v %) ethanol, 95% (v %) ethanol and acetone before the step (d).

In the example in which the first solvent is water and the second solvent is 95% (v %) ethanol, the method further includes the step of, (f) subjecting the solution of step (c) to column chromatography by eluting the column in sequence with n-hexane, ethyl acetate, and 70-80% (v %) ethanol before the step (d).

According to embodiments of the present disclosure, the respective products of steps (b) and (d), as well as the eluate collected from column chromatography may be concentrated or dried and use as a medicament for treating IBS.

Another aspect of the present invention pertains to a method for treating IBS in a subject in need of such treatment by administering to the subject an effective amount of the Alpinia spp. extract of this invention, particularly from the water extract of Alpinia spp., to ameliorate the symptoms of abnormal defecation and abdominal pain associated with IBS.

According to embodiments of the present disclosure, the extract of Alpinia oxyphylla includes, teucrenone or isalpinin; and at least one active compound selected from the group consisting of oxyphyllenodiol A, 6-α-hydroxy-7-epi-α-cyperone, 7-epi-teucrenone, and tectochrysin.

Among the compounds isolated from the Alpinia spp. extract, specifically from Alpinia oxyphylla extract, six of them have been confirmed to possess the capability of ameliorating the typical symptoms of IBS, i.e., abdominal pain associated with altered bowel movements; hence are potential lead compounds for use as an active agent for the preparation of a medicament for treating IBS. According to specific embodiment of the present disclosure, oxyphyllenodiol A, 6-α-hydroxy-7-epi-α-cyperone, 7-epi-teucrenone, teucrenone and tectochrysin respectively possess anti-abnormal defecation activity; wherease teucrenone and isalpinin are proved to possess anti-abdominal pain activity (or anti-visceral hypersensitivity). Accordingly, a combination of, teucrenone or isalpinin, and at least one compound selected from the group consisting of oxyphyllenodiol A, 6-α-hydroxy-7-epi-α-cyperone, 7-epi-teucrenone, and tectochrysin, would give rise to an extract that is capable of ameliorating the symptoms of IBS.

Many of the attendant features and advantages of the present disclosure will become better understood with reference to the following detailed description.

### BRIEF DESCRIPTION OF THE DRAWINGS

These and other features, aspects and advantages of the present invention will become better understood with reference to the following description, appended claims and the accompanying drawings, where:

FIG. 1A is a bar diagram illustrating the dose-dependent anti-abnormal defecation effect of the refined water extract of example 1.1.2 expressed in diarrhea score in accordance with one embodiment of the present invention, in which * represents p<0.05;

FIG. 1B is a bar diagram illustrating the dose-dependent anti-abnormal defecation effect of the refined water extract of example 1.1.2 expressed in the number of stools excreted in 30 min in accordance with one embodiment of the present invention, in which * represents p<0.05;

FIG. 1C is a bar diagram illustrating the anti-abnormal defecation effect of the refined water extract of example 1.1.3 expressed in diarrhea score in accordance with one embodiment of the present invention, in which * represents p<0.05;

FIG. 1D is a bar diagram illustrating the anti-abnormal defecation effect of the refined water extract of example 1.1.3 expressed in the number of stools excreted in 30 min in accordance with one embodiment of the present invention, in which * represents p<0.05;

FIG. 1E is a bar diagram illustrating the respective anti-abnormal defecation effects of the water extract paste of example 1.1.1.1 and the ethanol extract paste of example 1.2.1.1 expressed in diarrhea score in accordance with one embodiment of the present invention, in which * represents p<0.05;

FIG. 1F is a bar diagram illustrating the respective anti-abnormal defecation effects of the water extract paste of example 1.1.1.1 and the ethanol extract paste of example 1.2.1.1 expressed in the number of stools excreted in 30 min in accordance with one embodiment of the present invention, in which * represents p<0.05;

FIG. 2A is a bar diagram illustrating the dose-dependent anti-abnormal defecation effects of the water extract paste of example 1.1.1.1 expressed in diarrhea score in accordance with one embodiment of the present invention, in which N=10 mice/group, and * represents p<0.05;

FIG. 2B is a bar diagram illustrating the dose-dependent anti-abnormal defecation effects of the water extract paste of example 1.1.1.1 expressed in the number of stools excreted in 30 min in accordance with one embodiment of the present invention, in which * represents p<0.05;

FIG. 2C is a bar diagram illustrating the dose-dependent anti-abnormal defecation effects of the ethanol extract paste of example 1.2.1.1 expressed in diarrhea score in accordance with one embodiment of the present invention, in which * represents p<0.05;

FIG. 2D is a bar diagram illustrating the dose-dependent anti-abnormal defecation effects of the ethanol extract paste of example 1.2.1.1 expressed in number of stools excreted in 30 min in accordance with one embodiment of the present invention, in which * represents p<0.05;

FIG. 2E is a bar diagram illustrating the respective anti-abnormal defecation effects of the water extract paste of example 1.1.1.1 and the ethanol extract paste of example 1.2.1.1 expressed in diarrhea score in accordance with one embodiment of the present invention, in which * represents p<0.05;

FIG. 2F is a bar diagram illustrating the respective anti-abnormal defecation effects of the water extract paste of example 1.1.1.1 and the ethanol extract paste of example 1.2.1.1 expressed in the number of stools excreted in 30 min in accordance with one embodiment of the present invention, in which * represents p<0.05;

FIG. 2G is a bar diagram illustrating the respective anti-abnormal defecation effects of the refined water extract paste of example 1.1.1.1.1 expressed in diarrhea score in accordance with one embodiment of the present invention, in which * represents p<0.05;

FIG. 2H is a bar diagram illustrating the respective anti-abnormal defecation effects of the refined water extract paste of example 1.1.1.1.1 expressed in the number of stools excreted in 30 min in accordance with one embodiment of the present invention, in which * represents p<0.05;

FIG. 2I is a bar diagram illustrating the respective anti-abnormal defecation effects of the refined ethanol extract paste of example 1.2.1.1.2 expressed in diarrhea score in accordance with one embodiment of the present invention, in which * represents p<0.05;

FIG. 2J is a bar diagram illustrating the respective anti-abnormal defecation effects of the refined ethanol extract paste of example 1.2.1.1.2 expressed in the number of stools excreted in 30 min in accordance with one embodiment of the present invention, in which * represents p<0.05;

FIG. 2K is a bar diagram illustrating the respective anti-abnormal defecation effects of the acetone extract of example 1.3.1 and the ethyl acetate extract of example 1.4.1 expressed in diarrhea score in accordance with one embodiment of the present invention, in which * represents p<0.05;

FIG. 2L is a bar diagram illustrating the respective anti-abnormal defecation effects of the acetone extract of example 1.3.1 and the ethyl acetate extract of example 1.4.1 expressed in number of stools excreted in 30 min in accordance with one embodiment of the present invention, in which * represents p<0.05;

FIGS. 3A to 3C are line diagrams illustrating the anti-visceral hypersensitivity effect of (A) the water extract powders of example 1.1.2, (B) the water extract powders of example 1.1.3, and (C) granisetron in accordance with one embodiment of the present invention, in which *represent significant difference between PDC-1850 and 5-HTP rats, PDC-1918 and 5-HTP rats, granisetron and 5-HTP rats (p<0.05), and # represents significant difference between the baseline and 5-HTP rats (p<0.05);

FIGS. 4A and 4B are line diagrams illustrating the anti-visceral hypersensitivity effect of the water extract paste of example 1.1.1.1 at a dose of (A) 30 mg/kg and (B) 100 mg/kg in accordance with one embodiment of the present invention, in which * represents p<0.05 and # represents significant difference between the baseline and 5-HTP rats (p<0.05);

FIG. 4C is a line diagram illustrating the anti-visceral hypersensitivity effect of the refined water extract paste of example 1.1.1.1.1 at a dose of 200 mg/kg in accordance with one embodiment of the present invention;

FIGS. 5A, 5B and 5C are line diagrams illustrating the anti-visceral hypersensitivity effect of the ethanol extract paste of example 1.2.1.1 at a dose of (A) 100 mg/kg and (B) 300 mg/kg, and (C) granisetron at a dose of 10 μg/kg in accordance with one embodiment of the present invention, in which * represents p<0.05 and # represents significant difference between the baseline and 5-HTP rats (p<0.05);

FIG. 5D is a line graph illustrating the anti-visceral hypersensitivity effect of the refined ethanol extract paste of example 1.2.1.1.2 at a dose of 100 mg/kg in accordance with one embodiment of the present invention;

FIGS. 6A and 6B are line diagrams respectively illustrating the anti-visceral hypersensitivity effect of acetone extract of example 1.3.1 and the ethyl acetate extract of example 1.4.1 in accordance with one embodiment of the present invention, in which N=3 rats/group, and results from one particular rat are illustrated here;

FIG. 7A is a bar diagram illustrating the respective anti-abnormal defecation effects of the water extract paste of example 2.1 and the ethanol extract paste of example 2.2 expressed in diarrhea score in accordance with one embodiment of the present invention, in which * represents p<0.05;

FIG. 7B is a bar diagram illustrating the respective anti-abnormal defecation effects of the water extract paste of example 2.1 and the ethanol extract paste of example 2.2 expressed in the number of stools excreted in 30 min in accordance with one embodiment of the present invention, in which * represents p<0.05;

FIGS. 8A and 8B are line diagrams illustrating the anti-visceral hypersensitivity effect of the (A) water extract paste of example 2.1, and (B) ethanol extract paste of example 2.2 in accordance with one embodiment of the present invention;

FIG. 9A is a bar diagram illustrating the respective anti-abnormal defecation effects of the water extract of examples 3.1.1 and 3.1.2 expressed in diarrhea score in accordance with one embodiment of the present invention, in which * represents p<0.05;

FIG. 9B is a bar diagram illustrating the respective anti-abnormal defecation effects of the water extract of examples 3.1.1 and 3.1.2 expressed in the number of stools excreted in 30 min in accordance with one embodiment of the present invention, in which N=10 mice/group, and * represents p<0.05;

FIG. 9C is a line diagram illustrating the anti-visceral hypersensitivity effect of the ethanol extract paste of example 3.1.2 (i.e., PDC-1885) in accordance with one embodiment of the present invention, in which N=6 rats/group, and * represents p<0.05. # represents significant difference between the baseline and 5-HTP rats (p<0.05);

FIG. 10A is a bar diagram illustrating the respective anti-abnormal defecation effects of the water extract pastes of examples 3.1.3 and the ethanol extract of example 3.2.1.1 expressed in diarrhea score in accordance with one embodiment of the present invention, in which * represents p<0.05;

FIG. 10B is a bar diagram illustrating the respective anti-abnormal defecation effects of the water extract pastes of examples 3.1.3 and the ethanol extract of example 3.2.1.1 expressed the number of stools excreted in 30 min in accordance with one embodiment of the present invention, in which * represents p<0.05;

FIG. 10C is a line diagram illustrating the anti-visceral hypersensitivity effect of the water extract paste of example 3.1.3 (i.e., PDC-2469) in accordance with one embodiment of the present invention;

FIG. 10D is a line diagram illustrating the anti-visceral hypersensitivity effect of the ethanol extract paste of example 3.2.1.1 (i.e., PDC-2470) in accordance with one embodiment of the present invention

FIG. 11A is a bar diagram illustrating the respective anti-abnormal defecation effects of the water extract pastes of examples 4.1 and the ethanol extract of example 4.2 expressed in diarrhea score in accordance with one embodiment of the present invention, in which * represents p<0.05;

FIG. 11B is a bar diagram illustrating the respective anti-abnormal defecation effects of the water extract pastes of example 4.1 and the ethanol extract of example 4.2 expressed the number of stools excreted in 30 min in accordance with one embodiment of the present invention, in which * represents p<0.05;

FIG. 11C is a line diagram illustrating the anti-visceral hypersensitivity effect of the water extract paste of example 4.1 (i.e., PDC-2473) in accordance with one embodiment of the present invention;

FIG. 12A is a bar diagram illustrating the respective anti-abnormal defecation effects of the compounds of examples 5.1, 5.2, 5.3 and 5.4 expressed in diarrhea score in accordance with one embodiment of the present invention, in which N=5 mice/group, and * represents p<0.05;

FIG. 12B is a bar diagram illustrating the respective anti-abnormal defecation effects of the compounds of Examples 5.1, 5.2, 5.3 and 5.4 expressed in the number of stools excreted in 30 min in accordance with one embodiment of the present invention, in which N=5 mice/group, and * represents p<0.05;

FIG. 12C is a bar diagram illustrating the anti-abnormal defecation effect of the compound of Example 5.5 expressed in diarrhea score in accordance with one embodiment of the present invention, in which N=5 mice/group, * represents p<0.05; and

FIG. 12D is a bar diagram illustrating the anti-abnormal defecation effects of the compound of Example 5.5 expressed in the number of stools excreted in 30 min in accordance with one embodiment of the present invention, in which N=5 mice/group, represents p<0.05.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “treating” encompasses partially or completely preventing, ameliorating, mitigating and/or managing a symptom, a secondary disorder or a condition associated with irritable bowel syndrome (IBS). The term “treating” as used herein refers to application or administration of the Alpinia spp. extract prepared in accordance with the method of the present disclosure or compounds isolated therefrom to a subject, who has a symptom associated with IBS, a disorder secondary to IBS, or a predisposition toward IBS, with the purpose to partially or completely alleviate, ameliorate, relieve, delay onset of, inhibit progression of, reduce severity of, and/or reduce incidence of one or more symptoms, secondary disorders or features of IBS. Symptoms, secondary disorders, and/or conditions associated with IBS include, but are not limited to, pain, abdominal discomfort, abnormal stool frequency, abnormal stool consistency, diarrhea, and constipation. Treatment may be administered to a subject who exhibits only early signs of such symptoms, disorder, and/or condition for the purpose of decreasing the risk of developing the symptoms, secondary disorders, and/or conditions associated with IBS. Treatment is generally “effective” if one or more symptoms or clinical markers are reduced as that term is defined herein. Alternatively, a treatment is “effective” if the progression of a symptom, disorder or condition is reduced or halted.

The term “effective amount” as used herein refers to the quantity of a component or medicament which is sufficient to yield a desired “effective treatment” as defined hereinabove. The specific therapeutically effective amount will vary with factors such as the particular condition being treated, the physical condition of the patient (e.g., the patient's body mass, age, or gender), the type of mammal or animal being treated, the duration of the treatment, the nature of concurrent therapy (if any), and the specific formulations employed. An effective amount is also one in which any toxic or detrimental effects of the compound or composition are outweighed by the therapeutically beneficial effects. Effective amount may be expressed, for example, as the total mass of the medicament (e.g., in grams, milligrams or micrograms) or a ratio of mass of the medicament to body mass, e.g., as milligrams per kilogram (mg/kg). Persons having ordinary skills could calculate the human equivalent dose (HED) for the medicament (such as the Alpinia spp. extract or compounds isolated therefrom) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to an animal including the human species that is treatable with the Alpinia spp. extracts and/or compounds isolated therefrom in accordance with the methods of the present disclosure. The term “subject” is intended to refer to both the male and female gender unless one gender is specifically indicated, and may be any age, e.g., a child or adult.

As used herein, the term “fresh” refers to plant components that have not yet been processed, or only minimally processed (e.g., cut or sliced and/or packaged) after harvest and which are not preserved by substantive drying. Furthermore, the term “fresh” does not necessarily require a strict time-dependency. Rather, it is used solely to differentiate between dried plant components and non-dried plant components.

As used herein the term “dried” refers to a range of moisture contents typically observed when a plant component is dehydrated. The drying can occur by any means known in the art, including sun drying, oven drying and freeze drying. Moisture contents in dried plant components can range from 1 to 20% by weight, however, typical ranges are between 2 and 5%.

The term “extract of Alpinia spp.” or “Alpinia spp. extract” as used herein, refer to a composition prepared by contacting plant components from the Alpinia spp. plant, particularly Alpinia spp. plant selected from the group consisting of Alpinia oxyphylla, Alpinia zerumbet, Alpinia hainanensis and Alpinia galanga, with a suitable solvent in accordance with procedures described herein. As could be appreciated, the term “extract” encompasses crude extracts as well as processed or refined extract. Specifically, crude extracts are prepared by a simple extraction in which selected plant components are contacted with at least one extractant (i.e., extracting solvent). In some optional cases, the thus-obtained crude extracts are subject to one or more separation and/or purification steps to obtain purified, processed or refined extracts. The plant extract may be in liquid form, such as a solution, concentrate, or distillate; or it may be in solid form in which the solvent is removed, such as in paste, granulate or powder form.

The subject invention provides methods of treating a subject suffering from Irritable Bowel Syndrome (IBS), as well as the pharmaceutical preparations or the dietary supplements for use in practicing the subject methods.

One aspect of the present invention thus pertains to the discovery of active agents that are effective in treating IBS, particularly in ameliorating symptoms associated with IBS. The term “ameliorating” or “ameliorate” refers to any indicia of success in the treatment of a disorder or condition, including any objective or subject parameter such as abatement, remission or diminishing of symptoms or improvement in a patient's physical well-being, based on the results of a physical examination.

According to embodiments of the present invention, the method comprises administering to a subject diagnosed or suspected of having IBS an effective amount of an extract of Alpinia spp. or a compound purified therefrom. The subject may be diagnosed of having IBS using Rome II or Rome III process, which incorporates history, physical examination and basic investigations in IBS diagnosis.

In certain embodiments, the Alpinia spp. extract is given to the subject via oral administration. However, the present disclosure is not limited thereto.

According to embodiments of the present disclosure, the Alpinia spp. extract suitable for use in treating IBS is prepared in accordance with processes set forth in the Examples. According to examples of the present disclosure, plant components, particularly the fruits, collected from Alpinia spp. plants are minced and extracted with suitable solvent(s) to obtain crude extract(s). In certain embodiments of the present invention, fresh or dried fruits of Alpinia Oxyphylla are used to prepare the extract of this invention. The crude extract may subsequently be concentrated and/or dried (e.g., freeze-dried) to produce a crude extract powder or paste. Alternatively, it may be subject to further purification such as column chromatography or precipitation to produce a refined extract or a purified compound.

Examples of suitable solvent for extracting Alpinia spp. include, but are not limited to, a supercritical fluid (SFC) such as carbon dioxide, water, methane, ethane, propane, ethylene, propylene, methanol, ethanol and acetone; water; C₁₋₄ alcohol such as methanol, ethanol, propanol, n-butanol, iso-butanol, and ter-butanol; acetone; ethyl acetate; and n-hexane. According to certain embodiments of this invention, fresh or dried fruits of Alpinia spp. are minced and extracted with water; whereas in some other embodiments, they are extracted with an alcoholic solution consisting of water and 10-95% (v/v) ethanol. For example, the alcohol solution may be any of 10, 15, 20, 25, 30, 35, 40, 45, 50, 55, 60, 65, 70, 75, 80, 85, 90, and 95% (v/v) ethanol. In further embodiments, fresh or dried fruits of Alpinia spp. are minced and extracted with acetone. In still further embodiments, ethyl acetate is employed as the extraction solvent. According to embodiments of the present disclosure, the minced fruits of Alpinia spp. are mixed with the extraction solvent in a weight ratio from about 1:5 to about 1:50, such as 1:5, 1:7, 1:9, 1:10, 1:12, 1:15, 1:17, 1:20, 1:22, 1:25, 1:27, 1:30, 1:32, 1:35, 1:37, 1:40, 1:42, 1:45, 1:47 and 1:50; preferably from about 1:10 to 1:35, such as 1:5, 1:7, 1:10, 1:15, 1:20, 1:22, 1:25, 1:27, 1:30, 1:32, and 1:35. In other embodiments, SFC is employed as the extractant, which may or may not require the addition of a co-solvent, such as methanol or ethanol.

In optional embodiments, the crude extract is filtered, concentrated, and/or dried, and use as it is as a medicament to ameliorate symptoms associated with IBS.

In optional embodiment, the crude extract, such as the crude water extract, is further treated by adding a solvent with opposite polarity (e.g., 20-95% (v %) ethanol) and thereby generating a precipitate. The supernatant and/or precipitate may then be concentrated, and/or dried, which may also be used as a medicament for treating IBS.

In still another embodiment, the dried supernatant and/or precipitate obtained from the crude water extract may be subject to further purification by use of column chromatography. The column chromatography eluate is then concentrated and dried to produce a refined extract powders suitable for use in the preparation of a medicament for treating IBS. Non-limiting examples of the eluent for use in the column chromatography include, but are not limited to, water, 10-95% (v/v) ethanol, which includes, but are not limited to, 10%, 15%, 20%, 25%, 25%, 30%, 35%, 40%, 45%, 50%, 55%, 60%, 65%, 70%, 75%, 80%, 85%, 90% or 95% (v/v) ethanol; and acetone. In one example, the column is eluted in sequence, with water, 30% and 95% ethanol. In another example, the column is eluted in sequence, with 20%, 50%, and 95% ethanol. In other example, the column is eluted in sequence, with 20% and 95% ethanol, and acetone. In still another example, the column is eluted in sequence, with 40%, 70%, and 95% ethanol, and acetone.

Another aspect of the present invention pertains to a compound isolated from the Alpinia spp. extract prepared as described above, such as from the crude water extract paste of Alpinia Oxyphylla.

According to embodiments of the present disclosure, the Alpinia spp. extract is subject to a serial of column chromatography, such as silica gel chromatography and/or high pressure liquid chromatography (HPLC) to generate various fractions in accordance with procedures set forth in Examples of the present disclosure. Each fraction is then tested and confirmed the structure of the active compound(s) therein by spectra analysis, which includes but is not limited to, MS, ¹H-NMR, and ¹³C-NMR analysis. The compounds identified from each fraction are then tested for their effects on improving symptoms associated with IBS, i.e., abnormal defecation and abdominal pain. These compounds are potential lead compounds for developing medicaments suitable for treating IBS.

In specific examples, the identified compound is any of the following,

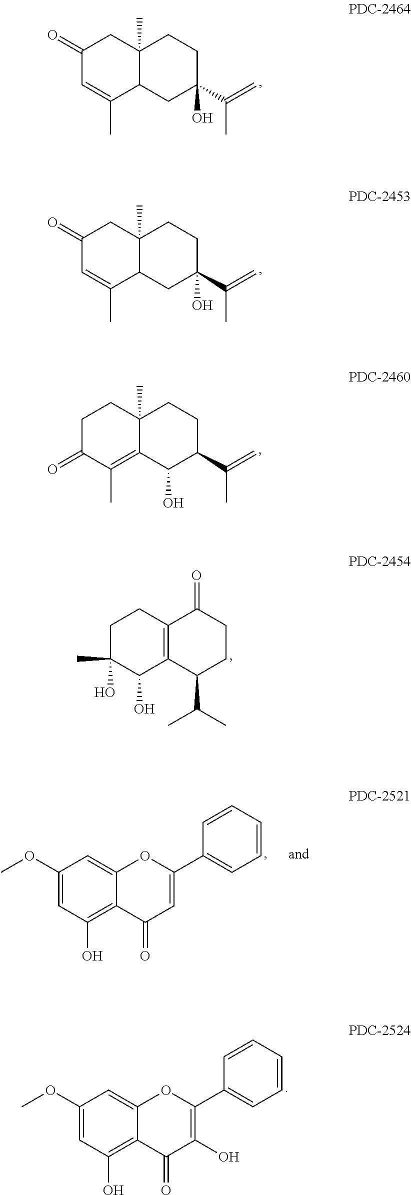

The present disclosure thus pertains to a pharmaceutical composition for treating IBS. In some embodiments, the Alpinia spp. extract or the active compounds of the present disclosure may be formulated into pharmaceutical compositions by combining with appropriate pharmaceutically acceptable carriers or excipients, and may be formulated into solid, semi-solid, or liquid forms, such as tablets, capsules, powders, granules, ointments, solutions, suppositories, and injections. As such, administration of the active compound can be achieved in various ways, including oral, buccal, rectal, parental, intraperitoneal, and etc. administration. In pharmaceutical dosage forms, Alpinia spp. extract or the active compound may be administered alone or in combination with other known pharmaceutically active agent to treat IBS. One of skilled person in the art is familiar with the various dosage forms that are suitable for use in each route. It is to be noted that the most suitable route in any given case would depend on the nature or severity of the disease or condition being treated.

In some embodiments, the pharmaceutical compositions of this disclosure are solid dosage forms for oral administration. Such solid dosage forms may be capsules, sachets, tablets, pills, lozengens, powders or granules. In such forms, the active ingredient such as the Alpinia spp. extract or any of the compounds described above is mixed with at least one pharmaceutically acceptable excipient. Any of the described solid dosage forms may optionally contain coatings and shells, such as enteric coatings, and coatings for modifying the release rate of any of the ingredients. Examples of such coatings are well known in the art. In one example, the pharmaceutical compositions of this disclosure are tablets such as quick-release tablets. In still another example, the pharmaceutical compositions of this disclosure are formulated into sustained release forms. In another example, the pharmaceutical compositions of this disclosure are powders that are encapsulated in soft and hard gelatin capsules.

In some embodiments, the pharmaceutical compositions of the present disclosure are liquid dosage forms for oral administration. The liquid formulation may further include a buffering agent to maintain a desired pH. The liquid dosage formulations may also be filled into soft gelatin capsules. For example, the liquid may include a solution, suspension, emulsion, micro-emulsion, precipitate or any desired liquid media carrying the Alpinia spp. extract or any of the compound as described above, or a pharmaceutically acceptable derivative, salt or solvate thereof, or a combination thereof. The liquid may be designed to improve the solubility of the Alpinia spp. extract or the active compound as described above to form a drug-containing emulsion or disperse phase upon release.

In some embodiments, the pharmaceutical compositions of this disclosure are formulations suitable for parenteral administration, such as administration by injection, which includes, but is not limited to, subcutaneous, bolus injection, intramuscular, intraperitoneal and intravenous injection. The pharmaceutical compositions may be formulated as isotonic suspensions, solutions or emulsions in oily or aqueous vehicles, and may contain formulatoary agents such as suspending, stabilizing or dispersing agents. Alternatively, the compositions may be provided in dry form such as powders, crystallines or freeze-dried solids with sterile pyrogen-free water or isotonic saline before use. They may be presented in sterile ampoules or vials.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Example 1

### Preparation of Extracts of Alpinia oxyphylla

### 1.1 Crude Water Extract

Dried, crushed fruits of Alpinia oxyphylla (purchased from Hainan, China) were boiled and refluxed in reverse osmotic (RO) water in a ratio of 1:10 of Alpinia oxyphylla to water (w/w) for about 1 hour to produce a crude extract. The crude water extract was sifted through a 350-mesh sieve so as to filter out the parts of Alpinia oxyphylla. Retain the filtrate, and the parts of Alpinia oxyphylla were subject to another water extraction in the same manner as previously described, combined the filtrate collected in both extractions to give a crude water extract.

1.1.1 Crude Water Extract Powders (PDC-2014)

The crude water extract of example 1.1 was concentrated by freeze-drying to yield crude water extract powders (PDC-2014).

1.1.1.1 Water Extract Paste (PDC-2363)

The crude water extract powders (PDC-2014) was further reconstituted in 95% (v %) ethanol, and filtered (No: 2 filter paper), the filtrate was then concentrated to dryness to give a water extract paste (PDC-2363).

1.1.1.1.1 Refined Water Extract Pastes A (PDC-2529) and B (PDC-2530)

The water extract paste (PDC-2363) was reconstituted in 20% (v %) ethanol, then loaded into HP-20 Diaion column for purification. The column was eluted in sequence, with 20% (vol) ethanol, 95% (vol) ethanol, and acetone. The eluate from 20% ethanol was concentrated to give a refined paste A (PDC-2529); whereas the eluates respectively collected from 95% ethanol and acetone were combined and concentrated to give a refined paste B (PDC-2530).

1.1.2 Refined Water Extract (PDC-1850)

95% (vol) ethanol was added to the crude water extract of example 1.1 in a 1:1 volume ratio, the mixture was then shifted through a 350-mesh sieve; the filtrate was centrifuged, concentrated, and freeze-dried to produce a paste (PDC-1850).

1.1.3 Refined Water Extract (PDC-1918)

95% (vol) ethanol was added to the crude water extract of example 1.1 in a 1:1 volume ratio, the mixture was then shifted through a 350-mesh sieve; the filtrate was centrifuged, and concentrated until its volume was reduced to ⅓ of its original volume.

Water was added to the concentrated water extract described above in a 1:1 volume ratio, and the mixture was then loaded onto a Diaion HP-20 column (Diaion, Mitsubishi Chemistry Inc.) in which the weight ratio of the concentrate to the dry column packing material is about 1:20. The column was sequentially eluted by 3 bed volumes of RO water, 1 bed volume of 30% (v/v) ethanol, and 3 bed volumes of 95% (vol) ethanol. The two alcoholic eluates were respectively collected and combined into one. The combined eluates were then concentrated under a reduced pressure until the ethanol was substantially removed, then the residues were freeze-dried to yield refined water extract pastes (PDC-1918).

### 1.2 Crude Ethanol Extract

Dried, crushed fruits of Alpinia oxyphylla (purchased from Hainan, China) were mixed with 50% (v %) ethanol in a ratio of 1:10 of Alpinia oxyphylla to ethanol (w/w), for about 1 hour to produce a crude ethanol extract. The crude ethanol extract was sifted through a 350-mesh sieve and the filtered out fruits of Alpinia oxyphylla were extracted with 50% (v %) ethanol one more time in the same manner as previously described, the solution collected in both extractions were combined to give a crude ethanol extract.

1.2.1 Crude Ethanol Extract Powders (PDC-1941)

The crude ethanol extract of example 1.2 was sifted through a 350-mesh sieve and the filtrate was concentrated under a reduced pressure until the solvent was substantially removed, then the residues were freeze-dried to yield crude ethanol extract powders (PDC-1941).

1.2.1.1 Ethanol Extract Paste (PDC-2364)

The crude ethanol extract powders of example 1.2.1 was reconstituted in 95% ethanol and filtered through a No: 2 filter paper, the filtrate was then concentrated to dryness to give an ethanol extract paste (PDC-2364).

1.2.1.1.1 Refined Ethanol Extract Pastes A (PDC-2371), B (PDC-2372), C (PDC-2373) and D (PDC-2374)

The ethanol extract paste (PDC-2364) was reconstituted in 40% (v %) ethanol, then loaded into HP-20 Diaion column for further purification. The column was eluted in sequence, with 40% (vol), 70% (vol) and 95% (vol) ethanol, followed by acetone. The eluates respectively collected from 40% and 70% ethanol were concentrated and freeze-dried to yield pastes A (PDC-2371) and B (PDC-2372); whereas the eluates respectively collected from 95% ethanol and acetone were concentrated to yield refined paste C (PDC-2373) and D (PDC-2374).

1.2.1.1.2 Refined Ethanol Extract Pastes E (PDC-2531) and F (PDC-2532)

The ethanol extract paste (PDC-2364) was reconstituted in 20% (v %) ethanol, then loaded into HP-20 Diaion column for further purification. The column was eluted in sequence, with 20% (vol), and 95% (vol) ethanol. The eluates were respectively concentrated and yield refined paste E (PDC-2531) and F (PDC-2532).

### 1.3 Crude Acetone Extract

The crude acetone extract of Alpinia oxyphylla was prepared in accordance with similar procedures described in example 1.2, except ethanol was replaced by acetone.

1.3.1 Crude Acetone Extract Paste (PDC-2479)

The crude acetone extract of example 1.3 was concentrated under a reduced pressure until the solvent was substantially removed, then the residues were freeze-dried to yield a crude acetone extract paste.

### 1.4 Crude Ethyl Acetate Extract

The crude ethyl acetate extract of Alpinia oxyphylla was prepared in accordance with similar procedures described in example 1.2, except ethanol was replace by ethyl acetate.

1.4.1 Crude Ethyl Acetate Extract Paste (PDC-2478)

The crude acetone extract of example 1.4 was concentrated under a reduced pressure until the solvent was substantially removed, then the residues were freeze-dried to yield a crude acetone extract paste.

### 1.5 Supercritical Fluid (SCF) Extract

In this particular example, Alpinia oxyphylla was extracted by supercritical fluid (e.g., carbon dioxide) with or without modification of a co-solvent (e.g., ethanol) under supercritical temperature and pressure.

1.5.1 SCF Extract A

Dried, crushed fruits of Alpinia oxyphylla were placed in the pressure cell, carbon dioxide was then pumped as a liquid at temperature below 5° C. into the heating zone of the pressure cell, in which the pressure and temperature were respectively set at 300 bar and 50° C. Then, sequentially expanded the fluid into the first and second separators to allow the extracted material to be separated from CO₂, in which the pressure and temperature in the first and second separators were respectively set at 60 and 45 bar, and 45 and 20° C. The extraction took about 150 minutes to complete, and thereby producing SCF extract A.

1.5.2 SCF Extract B

SCF extract B was prepared in accordance with the similar steps described in example 1.5.1, except a co-solvent, ethanol, was also included in the extraction procedures; and a SCF extract B was produced.

### Example 2

### Preparation of Extracts of Alpinia hainanensis

### 2.1 Water Extract Paste (PDC-2471)

The water extract paste of Alpinia hainanensis was prepared in accordance with similar procedures sequentially described in examples 1.1, 1.1.1, and 1.1.1.1.

### 2.2 Ethanol Extract Paste (PDC-2472)

The crude ethanol extract of Alpinia hainanensis was prepared in accordance with similar procedures sequentially described in examples 1.2, 1.2.1, 1.2.1.1.

### Example 3

### Preparation of Extracts of Alpinia galanga

### 3.1 Crude Water Extract

The crude water extract of Alpinia galanga was prepared in accordance with similar procedures described in example 1.1.

3.1.1 Crude Water Extract Powder (PDC-2147)

The crude water extract of example 3.1 was concentrated under a reduced pressure until a water extract powder was obtained.

3.1.2 Water Extract Pastes (PDC-1885)

95% (v %) ethanol was added into the crude water extract of example 3.1 in a 1:1 volume ratio and a precipitate was formed. The mixture was then centrifuged at a speed of 10,000 rpm for 5 minutes so as to remove the precipitate. The upper clear solution was collected and further concentrated under a reduced pressure until the solvent was substantially removed. The residues were further subject to freeze-drying to give water extract pastes.

3.1.3 Water Extract Pastes (PDC-2469)

The crude water extract powders of example 3.1.1 was reconstituted in 95% (v %) ethanol (1 g of the powders in 30 mL of ethanol), the mixture was ultra-sonicated for at least 1 hour, then filtered through No: 2 filter paper, the filtrate was then concentrated under a reduced pressure to give a water extract paste.

### 3.2 Crude Ethanol Extract

The crude ethanol extract of Alpinia galanga was prepared in accordance with similar procedures described in example 1.2.

3.2.1 Crude Ethanol Extract Powders

The crude ethanol extract of example 3.2 was concentrated under a reduced pressure until the solvent was substantially removed, then the residues were freeze-dried to yield crude ethanol extract powders.

3.2.1.1 Ethanol Extract Paste (PDC-2470)

The crude ethanol extract powders of example 3.2.1 was reconstituted in 95% ethanol and filtered through No: 2 filter paper, the filtrate was then concentrated to dryness to give an ethanol extract paste.

### Example 4

### Preparation of Extracts of Alpinia zerumbet

### 4.1 Water Extract Pastes (PDC-2473)

The crude water extract pastes of Alpinia zerumbet was prepared in accordance with similar procedures sequentially described in examples 1.1, 1.1.1 and 1.1.1.1.

### 4.2 Ethanol Extract Pastes (PDC-2474)

The ethanol extract pastes of Alpinia zerumbet was prepared in accordance with similar procedures sequentially described in examples 1.2, 1.2.1, and 1.2.1.1.

### Example 5

### Purification and Identification of Active Compounds from Alpinia oxyphylla of Example 1

Pure compounds were isolated and purified using chromatography, and their respective structures were subsequently confirmed and identified by MS and NMR.

5.1 Identification of 6-α-hydroxy-7-epi-α-cyperone (Compound PDC-2460)

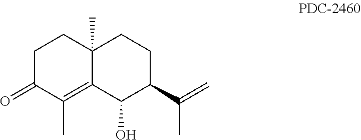 [SMILES: C=C(C)[C@@H]1CC[C@]2(C)CCC(=O)C(C)=C2[C@H]1O]

The water extract paste of example 1.1.1.1 was subject to silica gel filtration (Merck, Taiwan), by eluting the column with n-hexane and ethyl acetate, in which 7 fractions (FR-1, FR-2, FR-3, FR-4, FR-5, FR-6, and FR-7) were obtained. The second fraction (i.e., FR-2) out of the total 7 fractions was subsequently loaded onto a RP-18 gel column (Merck, Taiwan), and eluted with 80% MeOH, in which further 6 fractions (FR-21, FR-22, FR-23, FR-24, FR-25, and FR-26) were obtained. Combined both FR-25 and FR-1 fractions and loaded the combined fraction into another RP-18 gel column, and eluted with 80% MeOH to produce further 3 fractions (FR-25(1)1, FR-25(1)2, and FR-25(1)3). The FR-25(1)2 fraction was subject to another silica gel purification to give another 5 fractions (FR-25(1)21, FR-25(1)22, FR-25(1)23, FR-25(1)24, and FR-25(1)25). Compound PDC-2460 was purified from the third fraction, FR-25(1)23, by preparative HPLC analysis.

MS: Mw. 234 (Bruker MS) [M−H]⁻ (233). (C₁₅H₂₂C₂)

¹H NMR (CDCl₃, 600 MHz) δ 1.60 (1H, m, H-1), 1.83 (1H, m, H-1), 2.42 (1H, m, H-2), 2.62 (1H, m, H-2), 4.90 (1H, s, H-6), 2.53 (1H, br, s, H-7), 1.52 (1H, m, H-8), 2.19 (1H, m, H-8), 1.35 (1H, m, H-9), 1.47 (1H, m, H-9), 4.38 (1H, s, H-12), 4.81 (1H, s, H-12), 1.73 (3H, s, H-13), 1.38 (3H, s, H-14) and 1.88 (3H, s, H-15).

¹³C NMR (CDCl₃, 150 MHz) δ 38.84 (C-1), 34.17 (C-2), 199.90 (C-3), 132.11 (C-4), 159.94 (C-5), 69.60 (C-6), 47.64 (C-7), 18.57 (C-8), 35.02 (C-9), 35.03 (C-10), 145.25 (C-11), 111.46 (C-12), 22.96 (C-13), 25.72 (C-14), 10.43 (C-15).

5.2 Identification of 7-epi-teucrenone (Compound PDC-2453)

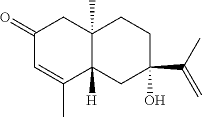 [SMILES: C=C(C)[C@]1(O)CC[C@]2(C)CC(=O)C=C(C)[C@H]2C1]

The fourth fraction (i.e., FR-4) out of the total 7 fractions of example 5.1 was loaded onto a RP-18 gel column (Merck, Taiwan), and eluted with 70% MeOH, in which further 5 fractions (FR-41, FR-42, FR-43, FR-44, and FR-45) were obtained. The FR-43 fraction was loaded into another RP-18 gel column, and eluted with 70% MeOH, to produce further 6 fractions (FR-431, FR-432, FR-433, FR-434, FR-435 and FR-436). Combined both FR-432 and FR-433 fractions and subjected the combined fractions to another silica gel purification to give further 7 fractions (FR-432(433)1, FR-432(433)2, FR-432(433)3, FR-432(433)4, FR-432(433)5, FR-432(433)6, and FR-432(433)7). Compound PDC-2453 was purified from the fourth fraction, FR-432(433)4, by preparative HPLC analysis.

MS: Mw. 234 (Bruker MS) [M−H]⁻ (233). (C₁₅H₂₂O₂)

¹H NMR (CDCl₃, 600 MHz) δ 2.10 (1H, d, J=15 Hz, H-1), 2.26 (1H, d, J=15 Hz, H-1), 5.86 (1H, br, s, H-3), 2.28 (1H, br, d, J=13 Hz, H-5), 1.52 (1H, m, H-6), 2.34 (1H, m, H-6), 1.75 (1H, m, H-8), 2.05 (1H, m, H-8), 1.42 (1H, m, H-9), 1.45 (1H, m, H-9), 5.08 (2H, br, s, H-12), 1.83 (3H, s, H-13), 0.95 (3H, s, H-14), 1.91 (3H, s, H-15).

¹³C NMR (CDCl₃, 150 MHz) δ 54.09 (C-1), 198.95 (C-2), 126.92 (C-3), 162.24 (C-4), 44.87 (C-5), 33.05 (C-6), 74.69 (C-7), 31.55 (C-8), 37.63 (C-9), 37.43 (C-10), 146.02 (C-11), 114.13 (C-12), 18.63 (C-13), 16.89 (C-14), 21.96 (C-15).

### 5.3 Identification of Teucrenone (Compound PDC-2464)

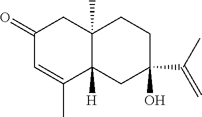 [SMILES: C=C(C)[C@@]1(O)CC[C@]2(C)CC(=O)C=C(C)[C@H]2C1]

The third fraction (i.e., FR-3) out of the total 7 fractions of example 5.1 was loaded onto a RP-18 gel column (Merck, Taiwan), and eluted with 70% MeOH, in which further 6 fractions (FR-31, FR-32, FR-33, FR-34, FR-35, and FR-36) were obtained. The FR-34 fraction was loaded into another RP-18 gel column, and eluted with 70% MeOH, to produce further 6 fractions (FR-341, FR-342, FR-343, FR-344, FR-345 and FR-346). Combined both FR-343 and FR-4 fractions, and subjected the combined fraction to another silica gel purification to give further 7 fractions (FR-341(4)1, FR-341(4)2, FR-341(4)3, FR-341(4)4, FR-341(4)5, FR-341(4)6, and FR-341(4)7). The FR-341(4)4 was added onto another RP-18 gel column, and eluted with 70% MeOH to produce further 6 fractions (FR-341(4)41, FR-341(4)42, FR-341(4)43, FR-341(4)44, FR-341(4)45 and FR-341(4)46). FR-341(4)44 fraction was further subject to silica gel purification, and compound PDC-2464 was purified by preparative HPLC from the sixth faction (i.e., FR-341(4)46).

MS: Mw. 234 (Bruker MS) [M−H]⁻ (233) (C₁₅H₂₂O₂)

¹H NMR (CDCl₃, 600 MHz) δ 2.26 (2H, d, J=3.5 Hz, H-1), 5.88 (1H, br, s, H-3), 2.93 (1H, m, H-5), 1.64 (1H, t, J=13.1 Hz, H-6), 1.78 (1H, m, H-6), 1.46 (1H, m, H-8), 1.92 (1H, m, H-8), 1.36 (1H, m, H-9), 1.84 (1H, m, H-9), 4.85 (1H, t, J=1.4 Hz, H-12), 5.07 (1H, s, H-12), 1.83 (3H, s, H-13), 0.87 (3H, s, H-14), 1.83 (3H, s, H-15).

¹³C NMR (CDCl₃, 150 MHz) δ 54.04 (C-1), 199.12 (C-2), 127.12 (C-3), 163.27 (C-4), 42.61 (C-5), 33.42 (C-6), 74.33 (C-7), 30.89 (C-8), 35.26 (C-9), 37.12 (C-10), 151.68 (C-11), 109.51 (C-12), 19.02 (C-13), 15.80 (C-14), 21.87 (C-15).

### 5.4 Identification of Oxyphellenodiol A (Compound PDC-2454)

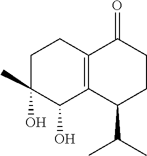 [SMILES: CC(C)[C@@H]1CCC(=O)C2=C1[C@H](O)[C@](C)(O)CC2]

The fourth fraction (i.e., FR-4) out of the total 7 fractions of example 5.1 was loaded onto a RP-18 gel column, and eluted with 70% MeOH, in which further 5 fractions (FR-41, FR-42, FR-43, FR-44, and FR-45) were obtained. The FR-44 fraction was loaded into another RP-18 gel column, and eluted with 70% MeOH, to produce further 5 fractions (FR-441, FR-442, FR-443, FR-444, and FR-445). Compound PDC-2454 was purified by preparative HPLC from the second faction (i.e., FR-442).

MS: Mw. 238 (Bruker MS) [M−H]⁻ (237) (C₁₄H₂₂O₃)

¹H NMR (CDCl₃, 600 MHz) δ 2.18 (1H, m, H-1), 2.45 (1H, m, H-1), 1.63 (1H, m, H-2), 1.75 (1H, m, H-2), 4.15 (1H, br, s, H-4), 2.60 (1H, m, H-6), 1.92 (2H, m, H-7), 2.26 (1H, m, H-8), 2.45 (1H, m, H-8), 2.18 (H, m, H-11), 0.86 (3H, d, J=6.9 Hz, H-12), 1.02 (3H, d, J=6.9 Hz, H-13), 1.18 (3H, s, H-14).

¹³C NMR (CDCl₃, 150 MHz) δ 21.40 (C-1), 32.08 (C-2), 72.31 (C-3), 75.08 (C-4), 157.81 (C-5), 40.00 (C-6), 22.21 (C-7), 34.94 (C-8), 199.95 (C-9), 132.41 (C-10), 29.76 (C-11), 19.11 (C-12), 21.53 (C-13), 21.72 (C-14).

### 5.5 Identification of Tectochrysin (Compound PDC-2521)

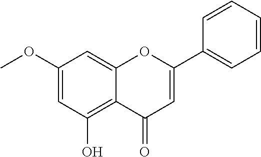 [SMILES: COc1cc(O)c2c(=O)cc(-c3ccccc3)oc2c1]

The first fraction (i.e., FR-1) out of the total 7 fractions of example 5.1 was loaded onto a RP-18 gel column, and eluted with 75% MeOH, in which further 5 fractions (FR-11, FR-12, FR-13, FR-14, and FR-15) were obtained. The FR-12 fraction was furthered subject to preparative HPLC analysis and compound PDC-2521 was obtained.

MS: 268 (Bruker MS) [M+H]⁺ (269) (C₁₆H₁₂O₄)

¹H NMR (CDCl₃, 600 MHz) δ 6.66 (1H, d, H-3), 6.36 (1H, d, J=2.1 Hz, H-6), 6.49 (1H, d, J=2.1 Hz, H-8), 7.87-7.89 (1H, m, H-2′), 7.49-7.55 (1H, m, H-3′), 7.49-7.55 (1H, m, H-4′), 7.49-7.55 (1H, m, H-5′), 7.87-7.89 (1H, m, H-6′), 3.87 (1H, s, OME).

¹³C NMR (CDCl₃, 150 MHz) δ 163.98 (C-2), 105.83 (C-3), 182.51 (C-4), 162.10 (C-5), 98.17 (C-6), 165.55 (C-7), 92.66 (C-8), 157.75 (C-9), 105.67 (C-10), 131.24 (C-1′), 126.28 (C-2′), 129.08 (C-3′), 131.85 (C-4′), 129.08 (C-5′), 126.28 (C-6′), 55.82 (OME).

### 5.6 Identification of Isalpinin (Compound PDC-2524)

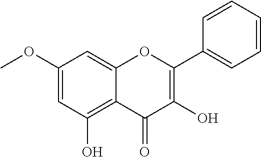 [SMILES: COc1cc(O)c2c(=O)c(O)c(-c3ccccc3)oc2c1]

The first fraction (i.e., FR-1) out of the total 7 fractions of example 5.1 was loaded onto a RP-18 gel column, and eluted with 75% MeOH, in which further 5 fractions (FR-11, FR-12, FR-13, FR-14, and FR-15) were obtained. The FR-12 fraction was furthered subject to preparative HPLC analysis and compound PDC-2524 was obtained.

MS: 284 (Bruker MS) [M+H]⁺ (285) (C₁₆H₁₂O₅)

¹H NMR (CDCl₃, 600 MHz) δ 6.36 (1H, d, J=2.1 Hz, H-6), 6.49 (1H, d, J=2.1 Hz, H-8), 8.19-8.17 (1H, m, H-2′), 7.46-7.51 (1H, m, H-3′), 7.46-7.51 (1H, m, H-4′), 7.46-7.51 (1H, m, H-5′), 8.19-8.17 (1H, m, H-6′), 3.87 (1H, s, OME).

¹³C NMR (CDCl₃, 150 MHz) δ 145.20 (C-2), 136.54 (C-3), 175.41 (C-4), 160.74 (C-5), 98.01 (C-6), 165.88 (C-7), 92.18 (C-8), 156.96 (C-9), 103.97 (C-10), 130.64 (C-1′), 127.57 (C-2′), 128.61 (C-3′), 130.28 (C-4′), 128.61 (C-5′), 127.57 (C-6′), 55.87 (OME).

### Example 6

### The Alpinia Spp. Extract Effectively Treat dl-5-Hydroxytryptophan (5-HTP) Induced Abnormal Defecation and/or Visceral Hypersensitivity

Serotonin is a major and significant monoamine-type neurotransmitter in the enteric nervous system. Ninety-five percent of the serotonin in the body is found in the gut, mainly in the enterochromaffin like (ECL) cells and in the enteric neurons; the remainder is found in the central nervous system (CNS). Serotonin may enhance sensitivity of visceral neurons projecting between the gastrointestinal tract and the CNS, and is involved in all integrated functions of the gut.

Since serotonin acts as an emerging neuromodulator in IBS, dl-5-hydroxytryptophan (5-HTP), which is converted into serotonin, was used as the IBS stimulant in this example to evaluate the efficacy of the Alpinia spp. extracts of Examples 1 to 4 on treating abnormal defecation symptoms associated with IBS. In the mean time, visceral hypersensitivity (VH), a defining factor in pathogenesis or IBS, as well as behavioral response animal models were respectively employed to evaluate the efficacy of the Alpinia spp. extracts of Examples 1 to 4 on treating the abdominal pain symptoms associated with IBS, using 5-HTP as the IBS stimulant.

The experimental procedures were approved by the Institutional Animal Care and Use Committee (IACUC) of Ricerca Biosciences, LLC (Taiwan), National Yang-Ming University (Taiwan) or Fu-Jen Catholic University (Taiwan), and conducted according to national animal welfare regulations.

Animal Model for Evaluating 5-HTP Induced Diarrhea and/or Abnormal Defecation

Male ICR mice (BioLASCO Taiwan Co., Ltd., Taiwan) were kept in an air-conditioned animal shelter at room temperature of 22° C. to 24° C. with controlled level of humidity (40% to 50%) in a 12-hour light-dark cycle. Each mouse weighed between 30 to 34 g in the beginning of the test. Tap water and standard laboratory rodent chow were provided ad libitum.

Each group consisted 10 mice, and each mice in the test group was given two doses of the Alpinia spp. extract of this invention (i.e., 500 mg/kg, 1,000 mg/kg, or 2,000 mg/kg) orally on day 1, and a third dose on day 2; and one dose of 5-HTP (i.p., 10 mg/kg) just 60 minutes after the administration of the third dose of the Alpinia spp. extract. In some occasions, the Alpinia spp. extracts of this invention were given intraperitoneally instead of orally. For comparison purpose, loperamide hydrochloride (oral; 2 mg/kg), ondansteron hydrochloride (oral; 2 mg/kg) or graniserton hydrochloride (oral; 2 mg/kg) were given once respectively to the control animals just 30 minutes prior to the administration of 5-HTP. Thirty minutes after 5-HTP injection, defecation of each animal was observed, with number of stools excreted for 30 min and diarrhea score respectively recorded accordingly. The diarrhea score was designed to describe consistency of stool using an arbitrary score scale from 0 to 3, with 0 representing solid form of stool, 1 representing loose form of stool, 2 representing partially liquid-like form of stool, and 3 representing watery form of stool.

### Animal Model for Evaluating 5-HTP Induced Visceral Hypersensitivity

Male Sprague-Dawley rats (BioLASCO Taiwan Co., Ltd., Taiwan) were kept in an air-conditioned animal shelter at room temperature of 22° C. to 24° C. with controlled level of humidity (40% to 50%) in a 12-hour light-dark cycle. Each rat weighed between 200 to 350 g in the beginning of the test. Tap water and standard laboratory rodent chow were provided ad libitum.

Unless specifically indicated, each group included 6 rats and experiments were conducted without fasting the animals. The plant extracts and positive control drugs were given to the animals either orally or intraperitoneally. On day 1, under ether anesthesia, an electromyogram (EMG) electrode was implanted onto the abdominal external oblique muscle of each animal; the animal was then allowed to recover from the implantation procedure for another 6 days. On day 8, animal was injected subcutaneously with a dose of 5-HTP (10 mg/kg) to induce visceral hypersensitivity and its response to colorectal distention (CRD) was recorded by EMG. Animals of the vehicle group were given a saline solution as well as the same vehicle of the corresponding experimental group orally; whereas animals in the experimental group were given Alpinia oxyphylla extract of Example 1, or other anti-VH medicament (e.g., 10 mg/kg of granisetron) orally at the specified time and dosage.

### Animal Model for Evaluating Chronic Visceral Hypersensitivity by Measuring Abnormal Withdrawal Reflex (AWR)

In this model, behavioral response was studied by measuring the AWR, which is an involuntary motor reflex similar to visceromotor reflex. The AWR was graded with the intensity of stimulus.

Male Sprague-Dawley rats (BioLASCO Taiwan Co., Ltd., Taiwan) were kept in an air-conditioned animal shelter at room temperature of 22° C. to 24° C. with controlled level of humidity (40% to 50%) in a 12-hour light-dark cycle. Each rat weighed between 200 to 350 g in the beginning of the test. Tap water and standard laboratory rodent chow were provided ad libitum. Unless specifically indicated, each group included 6 rats, and experiments were conducted without fasting the animals. The plant extracts and positive control drugs were given to the animals either orally or intraperitoneally.

Behavioral studies were assessed in according to previously described procedures (AL-CHAER et al., Gastroenterology 2000, 119:1276-1285). Briefly, the rats were then housed in small Lucite cubicles (20×8×8 cm) on an elevated Plexiglas platform and allowed to wake up and adapt (1 hr). Animal was injected subcutaneously with a dose of 5-HTP (10 mg/kg) to induce visceral hypersensitivity, and behavioral response was studied by measuring the AWR. Measurement of the AWR consisted of visual observation of the animal response by blinded observers and assignments of an AWR score: 0, no behavioral response; 1, brief head movement followed by immobility; 2, mild contraction of abdominal muscles without lifting the abdomen off the platform; 3, strong contraction of the abdominal muscles and lifting abdomen off the platform; 4, severe contraction of the abdominal muscle manifested by body arching and lifting of pelvic structures. Behavioral measurements were repeated by two different blinded observers.

All results are expressed as means±STD; n refers to animals in each group. Differences between animals of each group were compared by one-way

ANOVA followed by Dunnett's t test. A p value less than or equal to 0.05 was considered to be statistically significant.

6.1 Alpinia oxyphylla of Example 1 is Effective in Treating the Abnormal Defecation and Abdominal Pain Associated with IBS

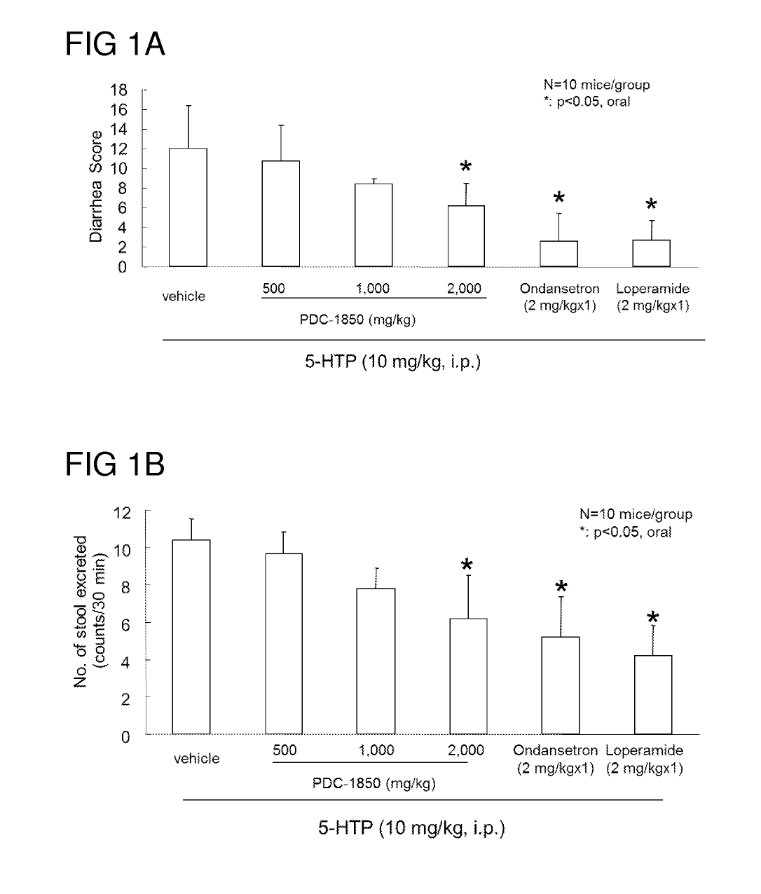

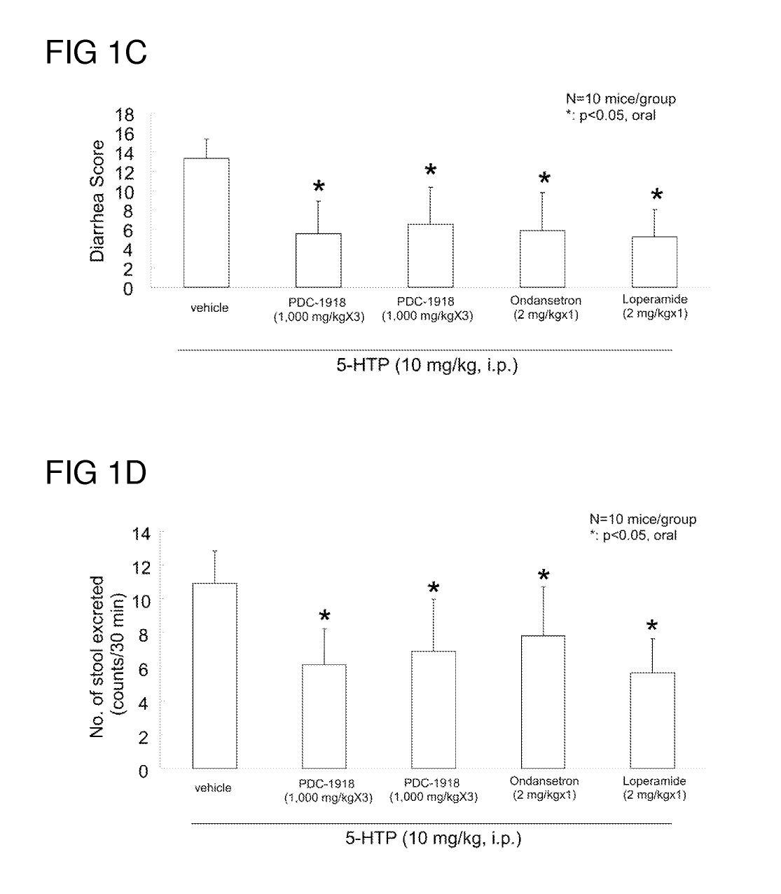

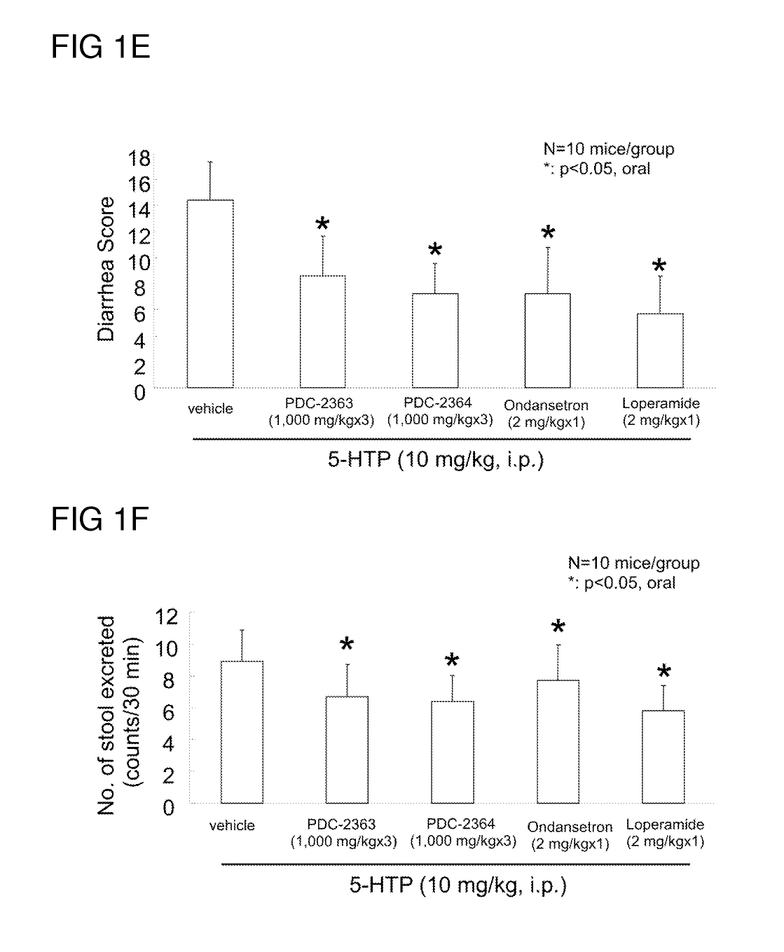

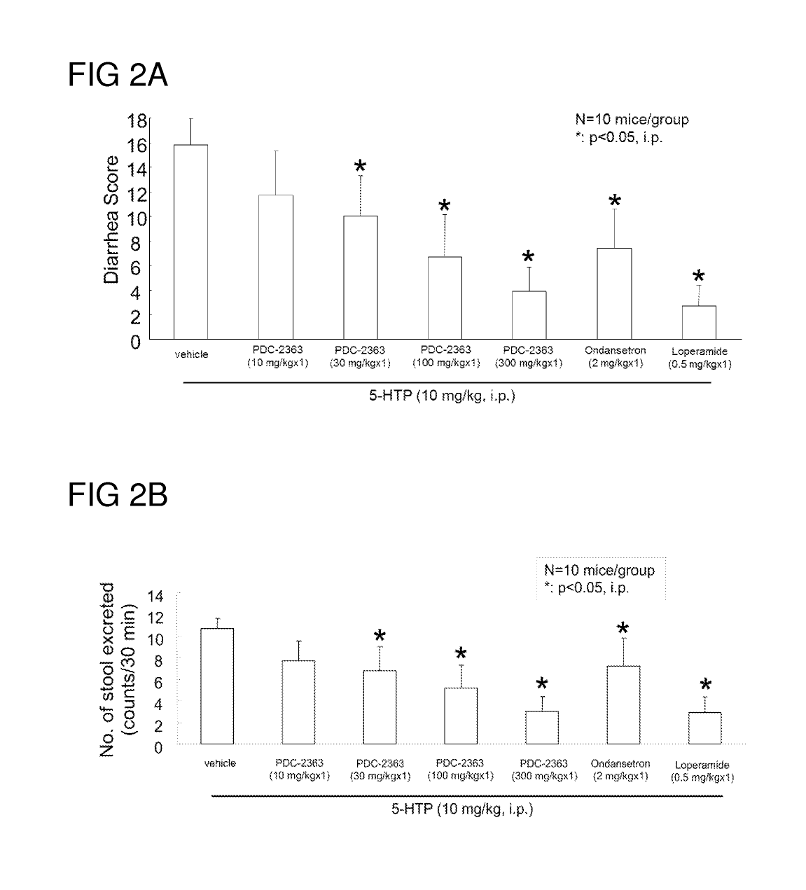

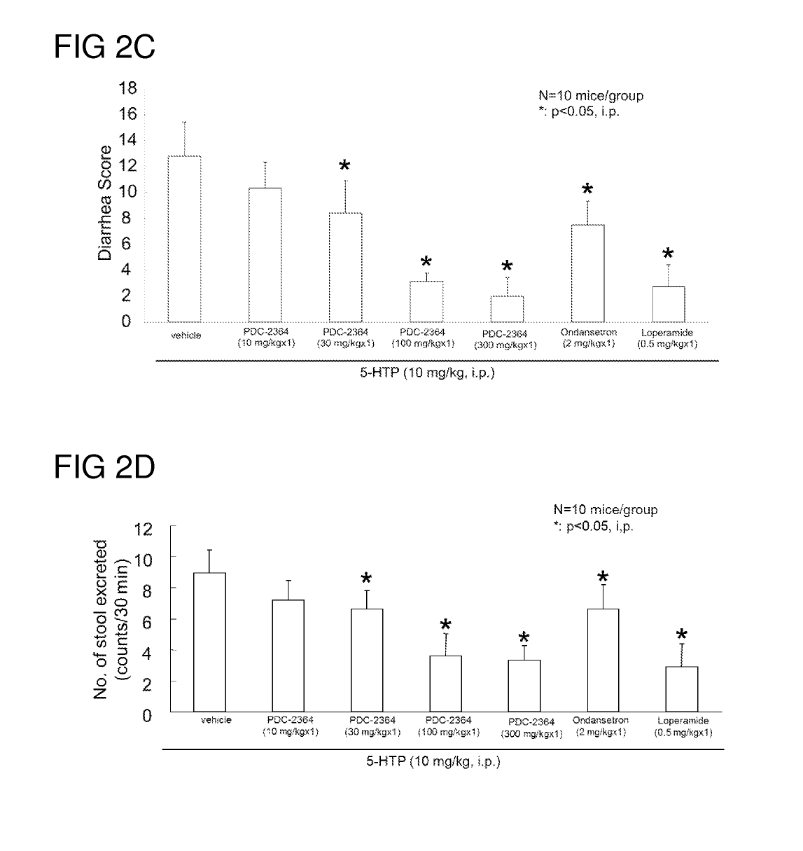

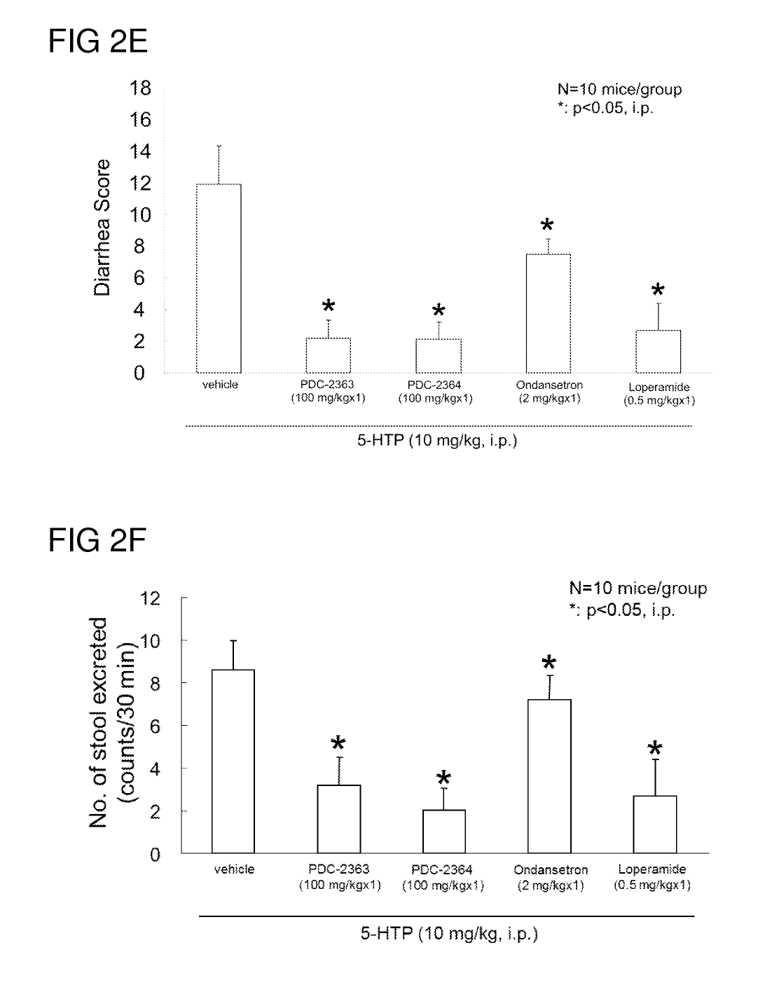

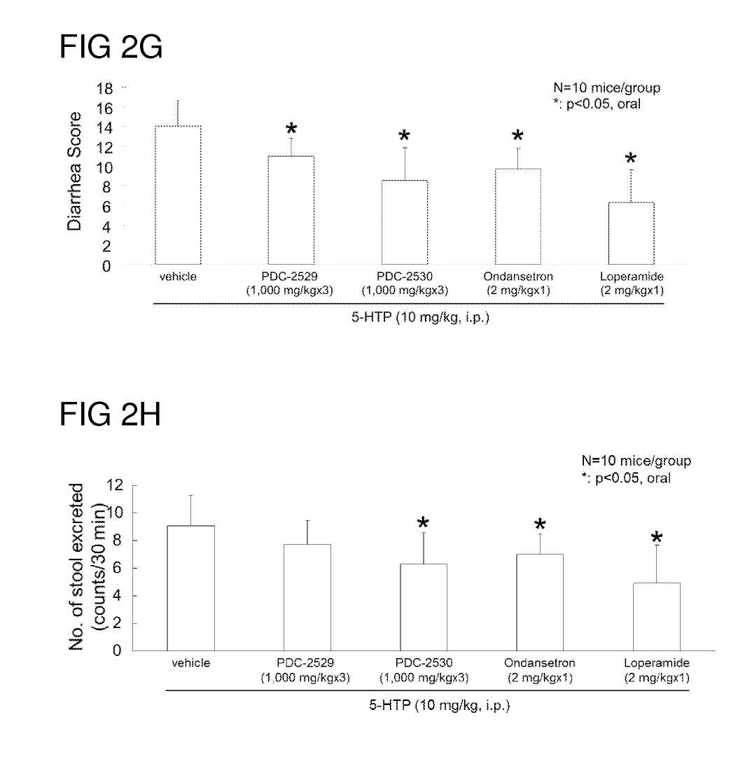

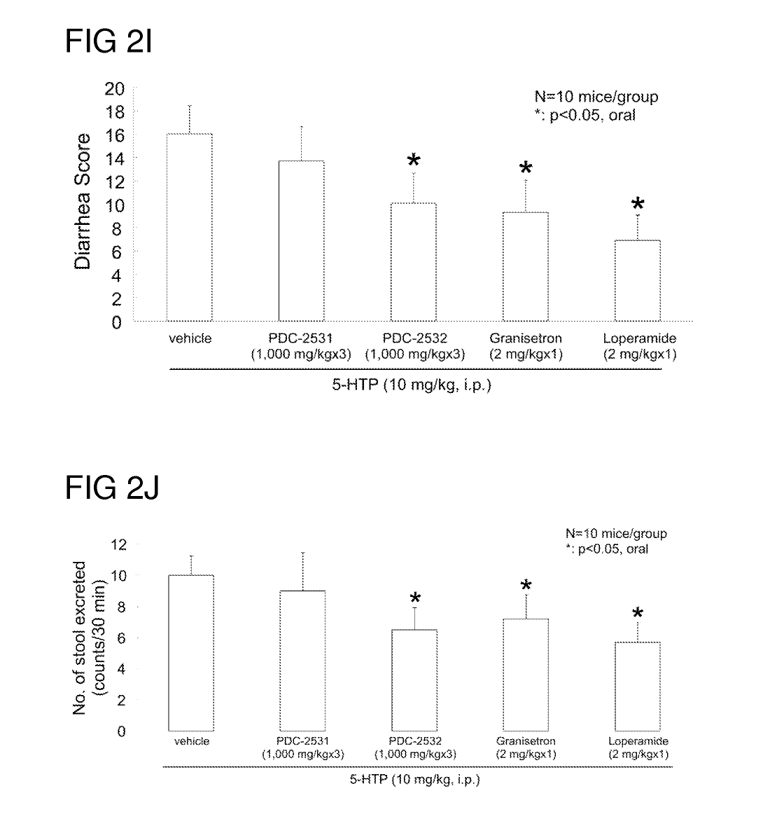

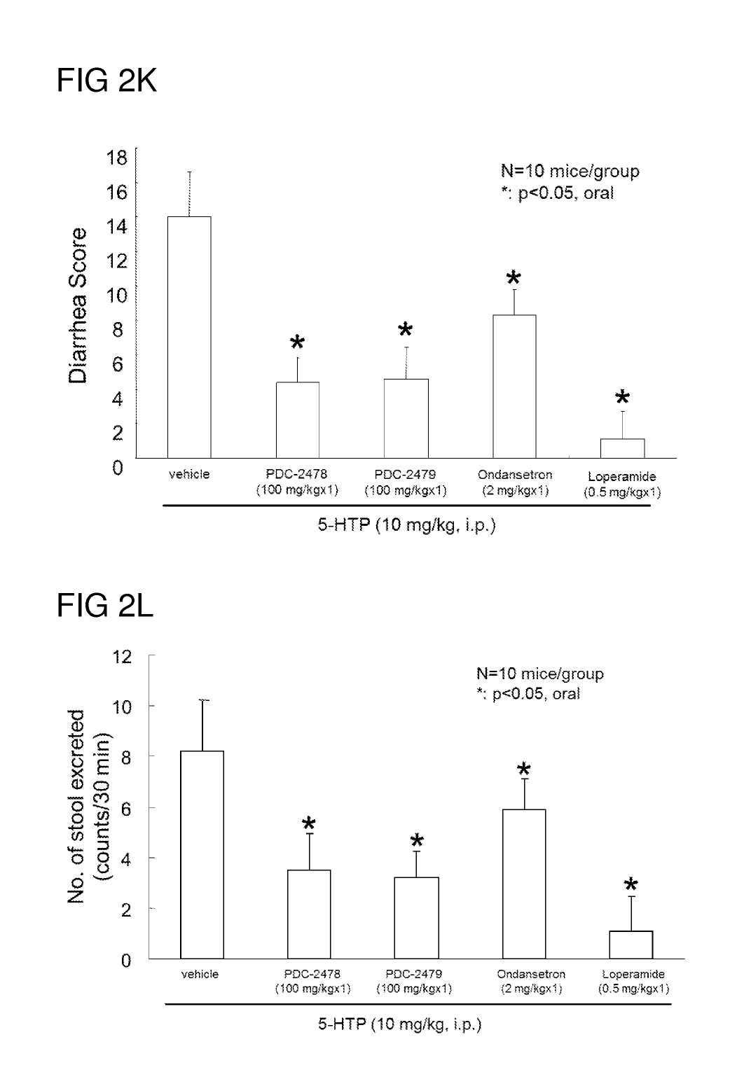

The effects of Alpinia oxyphylla of Example 1 on treating the abnormal defecation associated with IBS are illustrated in FIGS. 1 and 2. The effects were evaluated respectively by measuring the diarrhea score and counting the number of stools excreted in 30 min. As evident from FIG. 1, the water and alcoholic extracts of examples 1.1.2, 1.1.3, 1.1.1.1, 1.1.1.1.1, 1.2.1.1, and 1.2.1.1.2 are effective in ameliorating the abnormal defecation associated with IBS when given orally at a minimum dose of 1,000 mg/kg, as comparable to that of an opioid receptor agonist-loperamide or a 5-HT₃ receptor antagonist (e.g., ondansetron or granisetron). The improvement in abnormal defecation associated with IBS is more significant when the Alpinia oxyphylla extract of example 1.1.1.1 were given intraperitoneally, in which a low dose of 30 mg/kg is sufficient to exert anti-abnormal defecation action, with significant effect being observed at 100 mg/kg, about 10 times lower than that required when given orally (FIGS. 2A to 2J). Similarly, acetone extract or ethyl acetate extract of Alpinia oxyphylla were also capable of ameliorating the abnormal defecation associated with IBS (FIGS. 2K and 2L).

Anti-abnormal defecation effects of the extracts of example 1 were also compared with another commonly prescribed anti-spasmodic agent, e.g., mepenzolate, in amelioating symptoms associated with IBS, and both the extracts of example 1.1.1.1 and 1.2.1.1 were more effective in relieving relevant symptoms than that of mepenzolate (50 mg/kg, given orally 3 times) (results not shown).

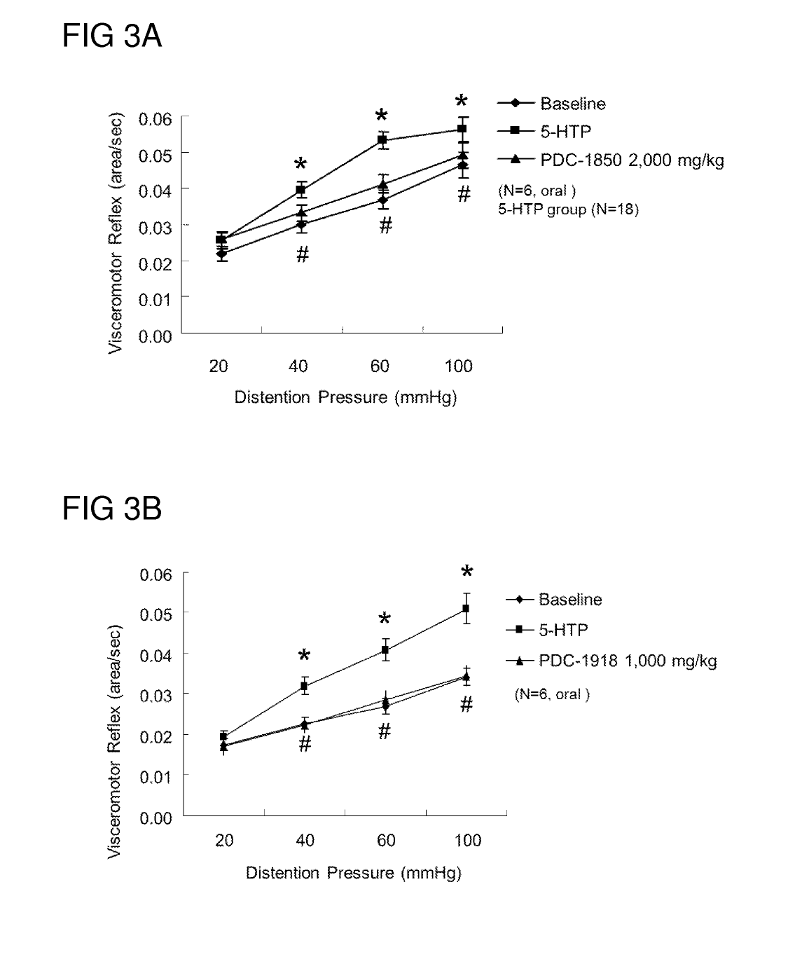

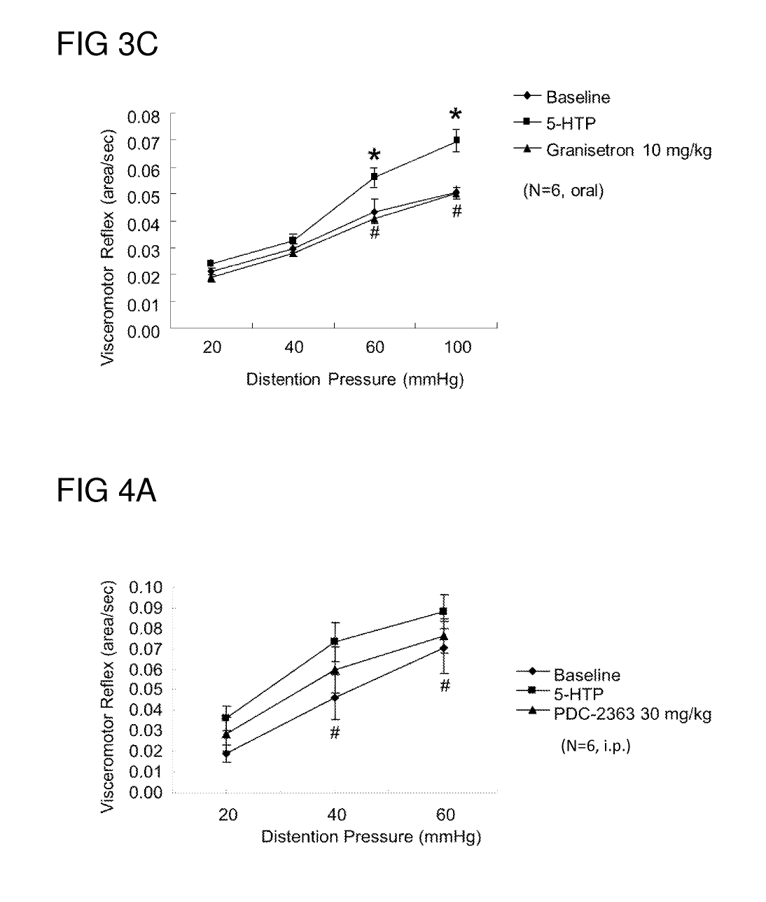

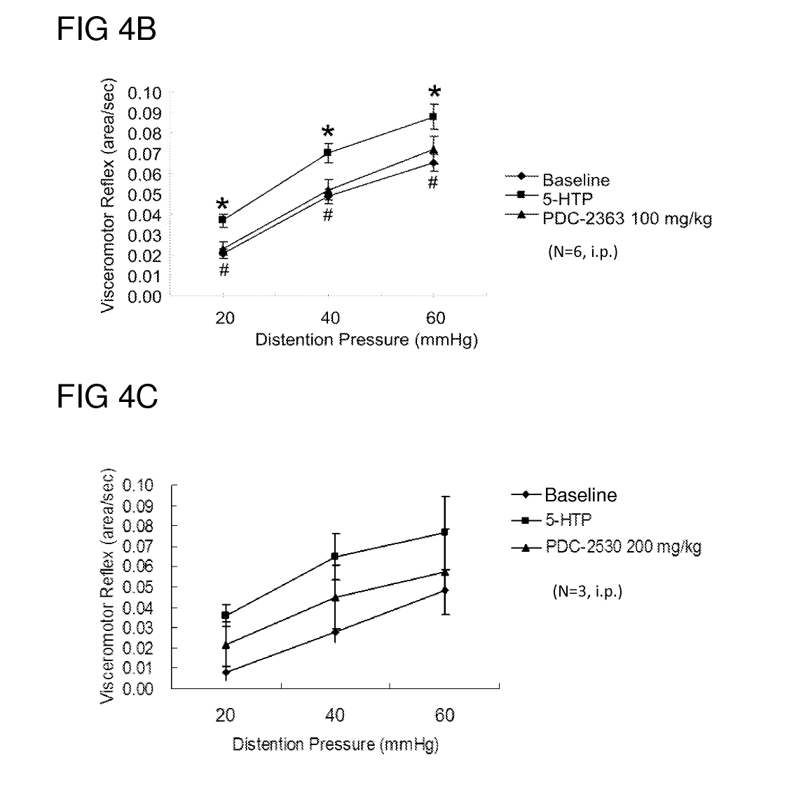

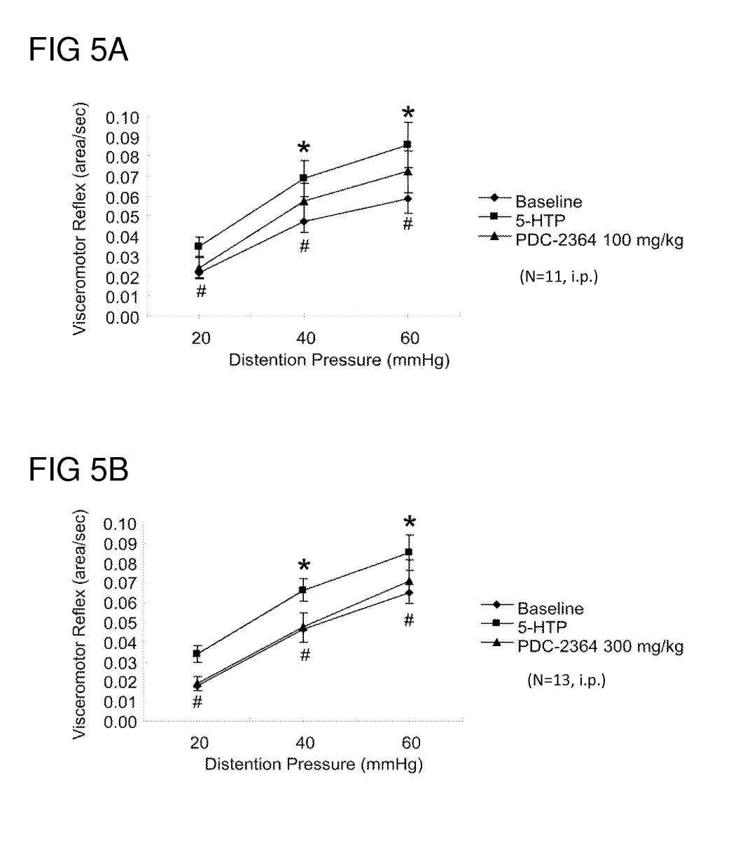

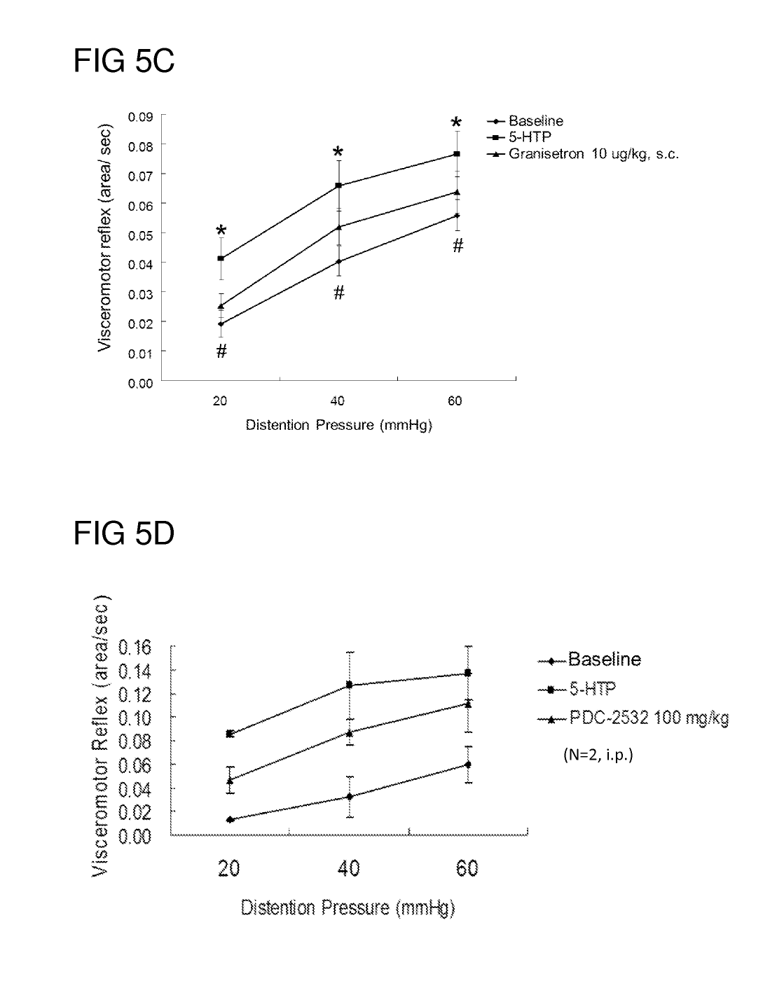

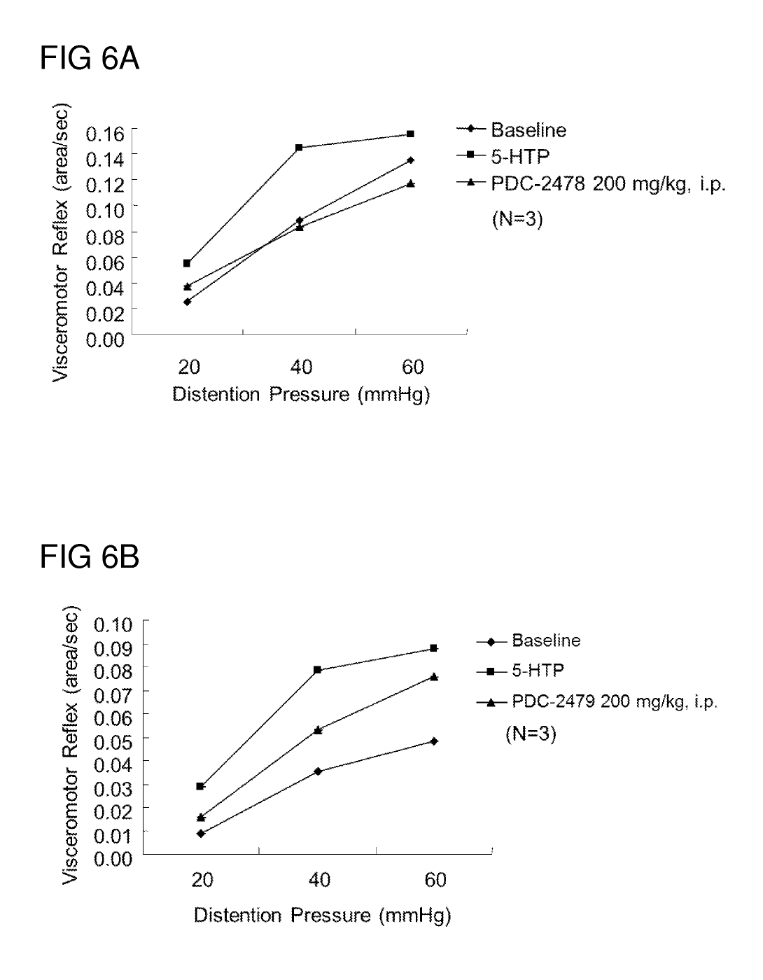

The anti-abdominal pain effects of the Alpinia oxyphylla extract of example 1 are illustrated in FIGS. 3 to 6. As depicted in FIGS. 3 and 4, the refined water extract powders of example 1.1.2 (i.e., PDC-1850) and example 1.1.3 (i.e., PDC-1918), the water extract paste of example 1.1.1.1 (i.e., PDC-2363) and the refined water extract paste of example 1.1.1.1.1 (i.e., PDC-2530) are all capable of reducing 5-HTP induced visceral hypersensitivity (i.e., as compared with the vehicle control), with its effect even more significantly than that of a 5-HT₃ receptor antagonist (e.g., granisetron or ondansetron). Similarly, the 50% ethanol extract of Alpinia oxyphylla (i.e., example 1.2.1.1 or PDC-2364; and example 1.2.1.1.2 or PDC-2532) (FIGS. 5A, 5B and 5D), as well as the EA (i.e., example 1.4.1 or PDC-2478) and acetone extract (i.e., example 1.3.1 or PDC-2479) of Alpinia oxyphylla (FIGS. 6A and 6B) respectively exhibited the same anti-5-HTP induced visceral hypersensitivity effect.

Behavioral responses as measured by AWR scores are summarized in Tables 1 and 2.

TABLE 1

| | | | body arching and |
|---|---|---|---|
| | | | lifting of pelvic |
| Group | Dose/route | lifting abdomen | structures |
| Sham (n = 5) | — | 0.12 ± 0.04 | 0.14 ± 0.09 |
| Vehicle (n = 5) | — (10 mL/kg) | 3.04 ± 0.66 | 2.59 ± 0.49 |
| The extract of | 30 mg/kg | 1.87 ± 0.32* | 1.93 ± 0.55* |
| Example 1.1.1.1 | i.p. | | |
| (PDC-2363) | | | |
| (n = 3) | | | |
| The extract of | 100 mg/kg | 1.46 ± 0.31* | 1.36 ± 0.38* |
| Example 1.1.1.1 | i.p. | | |
| (PDC-2363) | | | |
| (n = 5) | | | |
| 0.9% saline (n = 5) | — (10 mL/kg) | 2.92 ± 0.49 | 2.74 ± 0.54 |
| Granisetron | 100 μg/kg | 2.80 | 1.90 |
| (n = 1) | i.p. | | |

*means significant differences between the extract of example 1.1.1.1, granisetron and 5-HTP rat groups (p < 0.05).

TABLE 2

| | | | body arching and |
|---|---|---|---|
| | | | lifting of pelvic |
| Group | Dose/route | lifting abdomen | structures |
| Sham (n = 6) | — | 0.10 ± 0 | 0.10 ± 0 |
| 0.9% saline (n = 2) | — | 2.70 ± 0.14 | 2.70 ± 0.28 |
| Vehicle (n = 6) | Oral | 2.37 ± 0.12 | 2.58 ± 0.15 |
| (2% MC) | | | |
| The extract of | 250 mg/kg | 2.25 ± 0.07 | 2.45 ± 0.07 |
| Example 1.2.1.1.2 | Oral | | |
| (PDC-2532) | | | |
| (n = 2) | | | |
| The extract of | 500 mg/kg | 0.35 ± 0.07* | 0.40 ± 0.07* |
| Example 1.2.1.1.2 | Oral | | |
| (PDC-2532) | | | |
| (n = 2) | | | |
| The extract of | 1,000 mg/kg | 0.25 ± 0.07* | 0.25 ± 0.07* |
| Example 1.2.1.1.2 | Oral | | |
| (PDC-2532) | | | |
| (n = 2) | | | |

*means significant differences between the extract of example 1.2.1.1.2, granisetron and 5-HTP rat groups (p < 0.05).

As indicated in Table 1, AWR scores in rats treated with the water extract paste of example 1.1.1.1 (i.e., PDC-2363) are significantly lower as compared with those of the control rats or rats that received granisetron treatment. It is evident that the Alpinia oxyphylla extract of example 1.1.1.1 is effective in reducing the abdominal pain.

Similarly, in Table 2, AWR scores in rats treated with the refined ethanol extract paste of example 1.2.1.1.2 (i.e., PDC-2532) are significantly lower as compared with those of the control rats. It is evident that the refined ethanol extract paste of Alpinia oxyphylla (i.e., example 1.2.1.1.2) is effective in reducing the abdominal pain.

Taken together, these results indicate that Alpinia oxyphylla extracts prepared by the method described hereinabove are useful for treating symptoms associated with IBS, particularly, abnormal defecation and abdominal pain.

6.2 Alpinia hainanensis Extracts of Example 2 are Effective in Treating the Abnormal Defecation and Abdominal Pain Associated with IBS

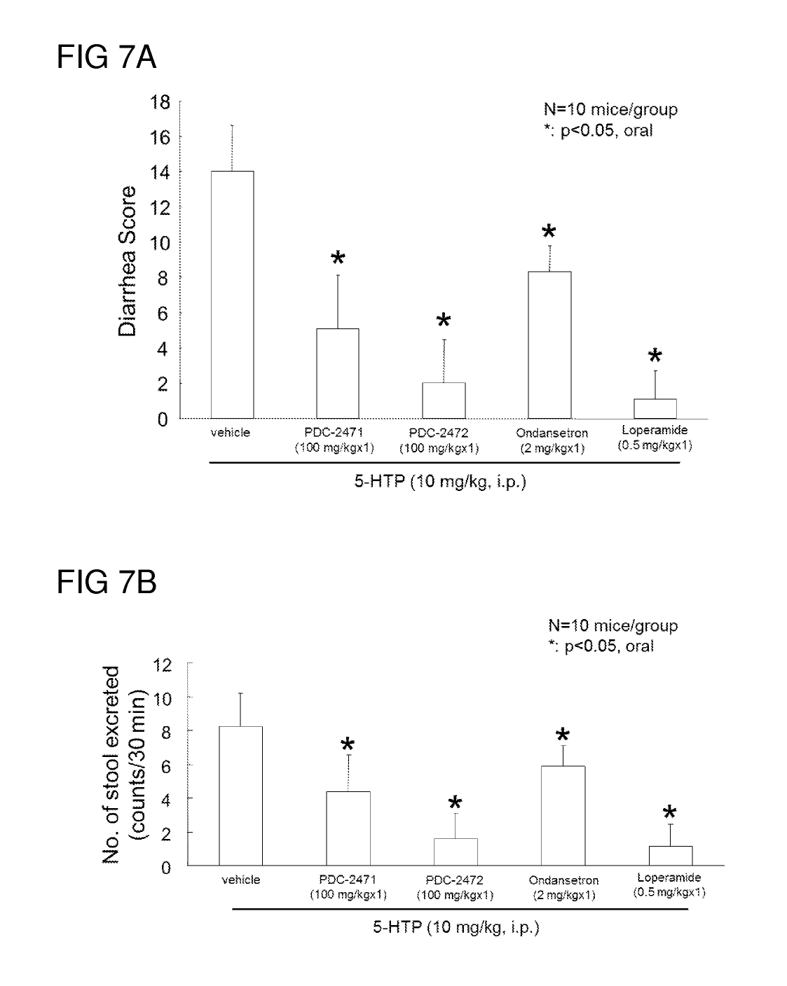

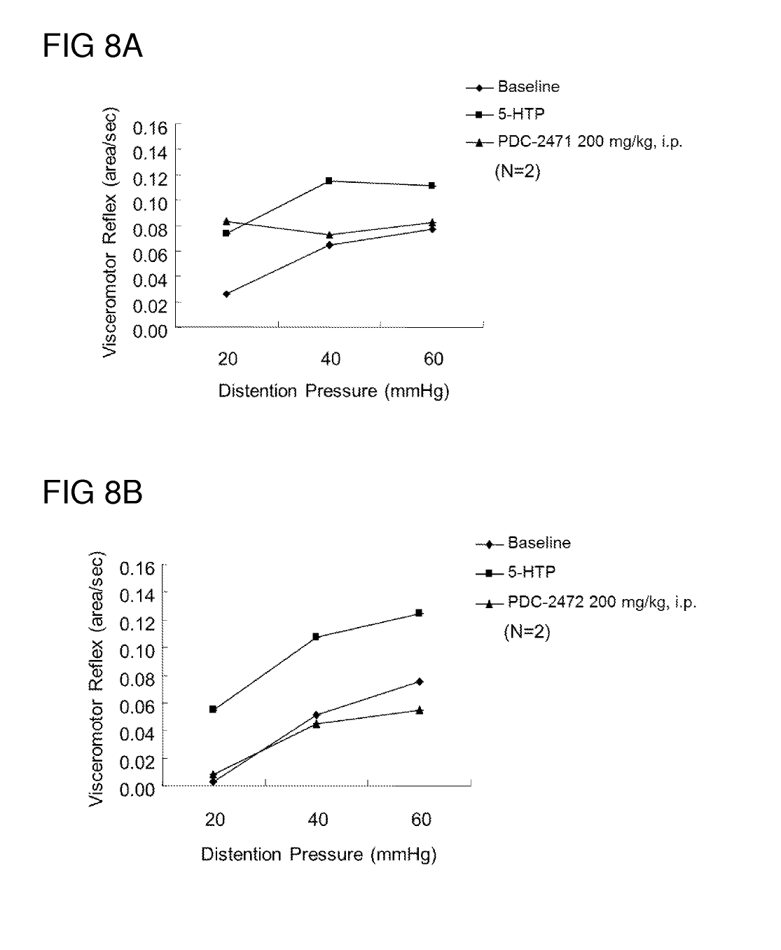

The effects of Alpinia hainanensis extracts of Example 2 on treating the symptoms associated with IBS are evaluated in accordance with similar procedures described above. Results are illustrated in FIGS. 7 and 8. Both the water (i.e., PDC-2471) and ethanol extract (i.e., PDC-2472) of Alpinia hainanensis were capable of lowering the diarrhea score and/or the number of stools excreted in IBS animals, as well as 5-HTP induced visceral hypersensitivity as compared with those of the control animals (FIGS. 8A and 8B).

6.3 Alpinia galanga Extracts of Example 3 are Effective in Treating the Abnormal Defecation and Abdominal Pain Associated with IBS

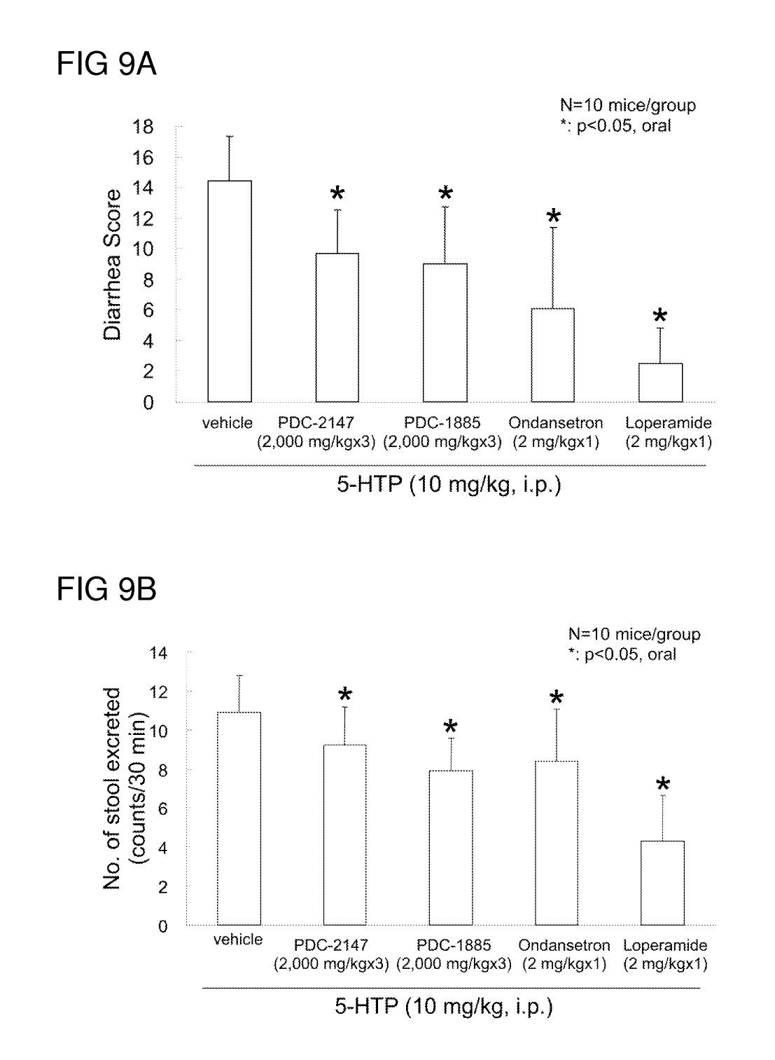

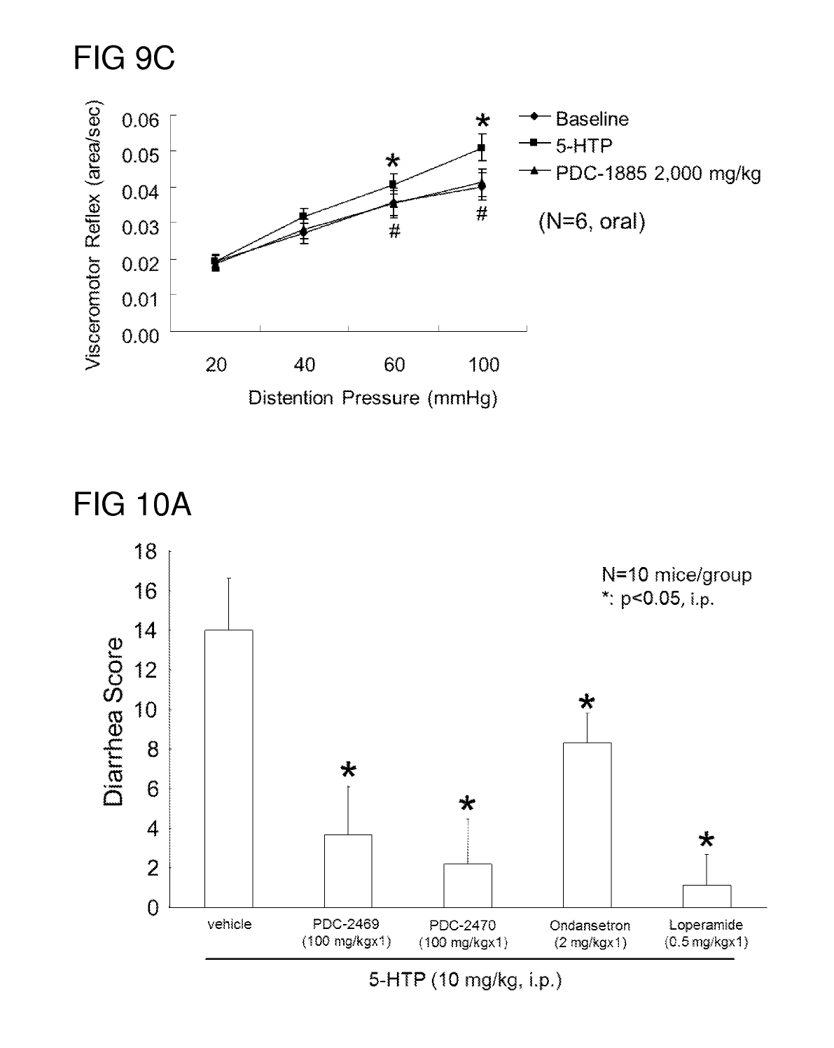

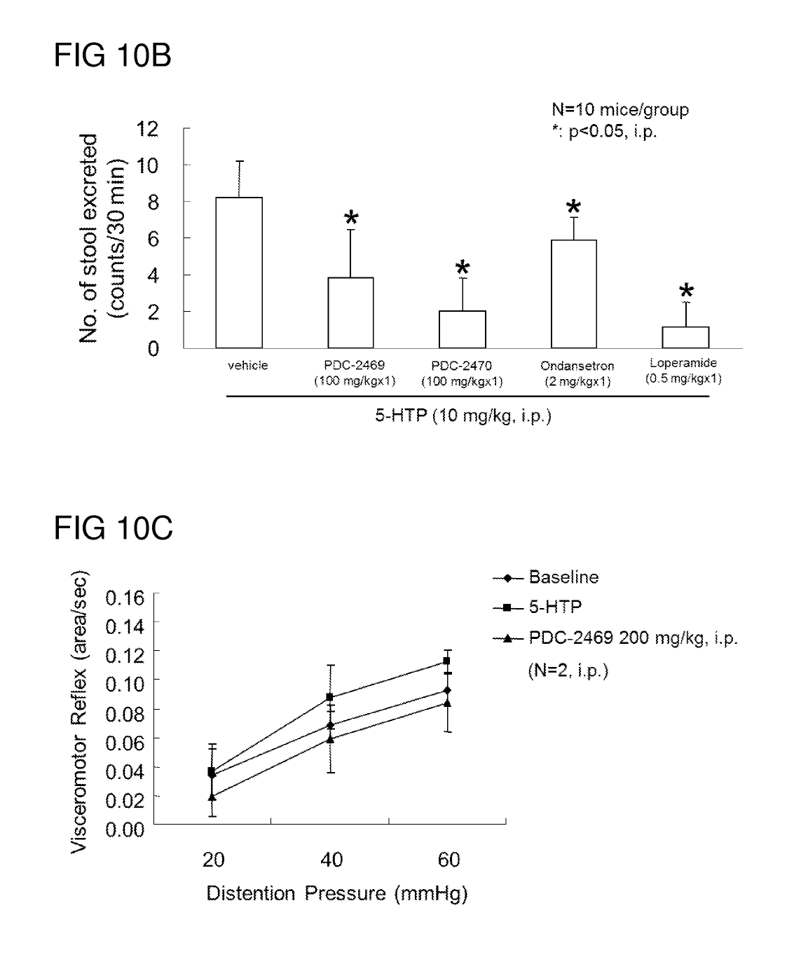

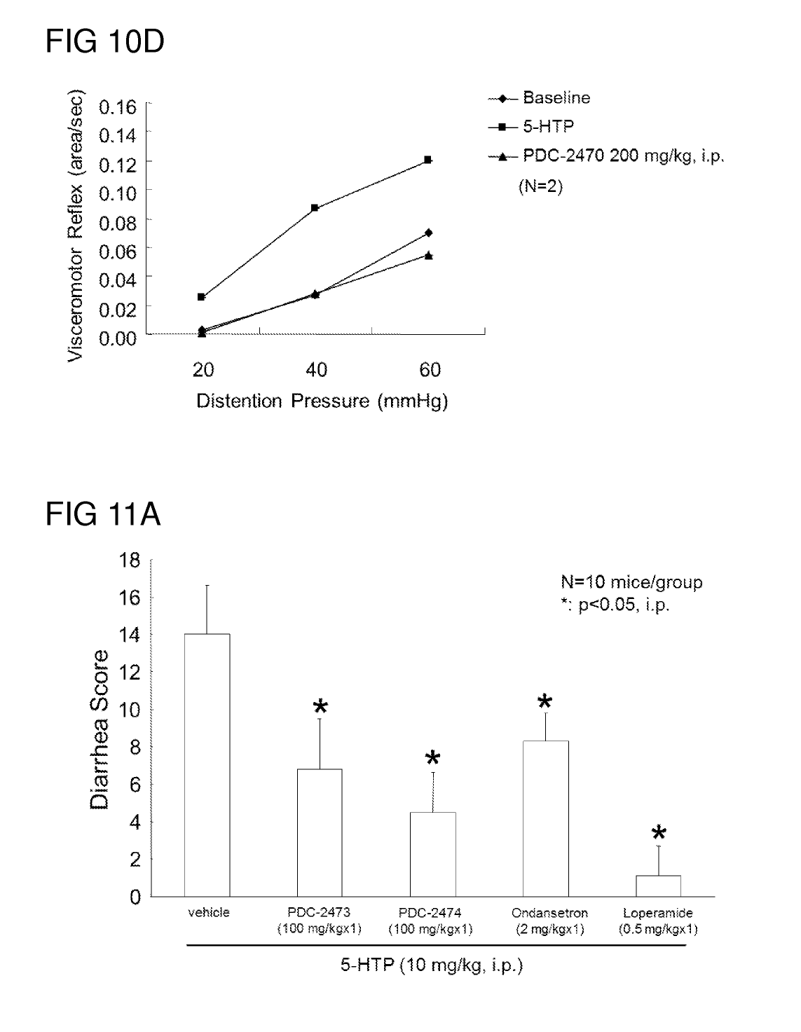

Effects of the Alpinia galanga extracts of Example 3 on symptoms associated with IBS are depicted in FIGS. 9 and 10. The water extracts of Alpinia galanga (i.e., PDC-2147 and PDC-1885) were capable of lowering the diarrhea score and/or the number of stools excreted in IBS animals (see FIGS. 9A and 9B), as well as the 5-HTP induced visceral hypersensitivity, as compared with that of the control animals (FIG. 9C). Similar effects were also observed in the ethanol extracts of Alpinia galanga (FIG. 10).

6.4 Alpinia zerumbet Extracts of Example 4 are Effective in Treating the Abnormal Defecation and Abdominal Pain Associated with IBS

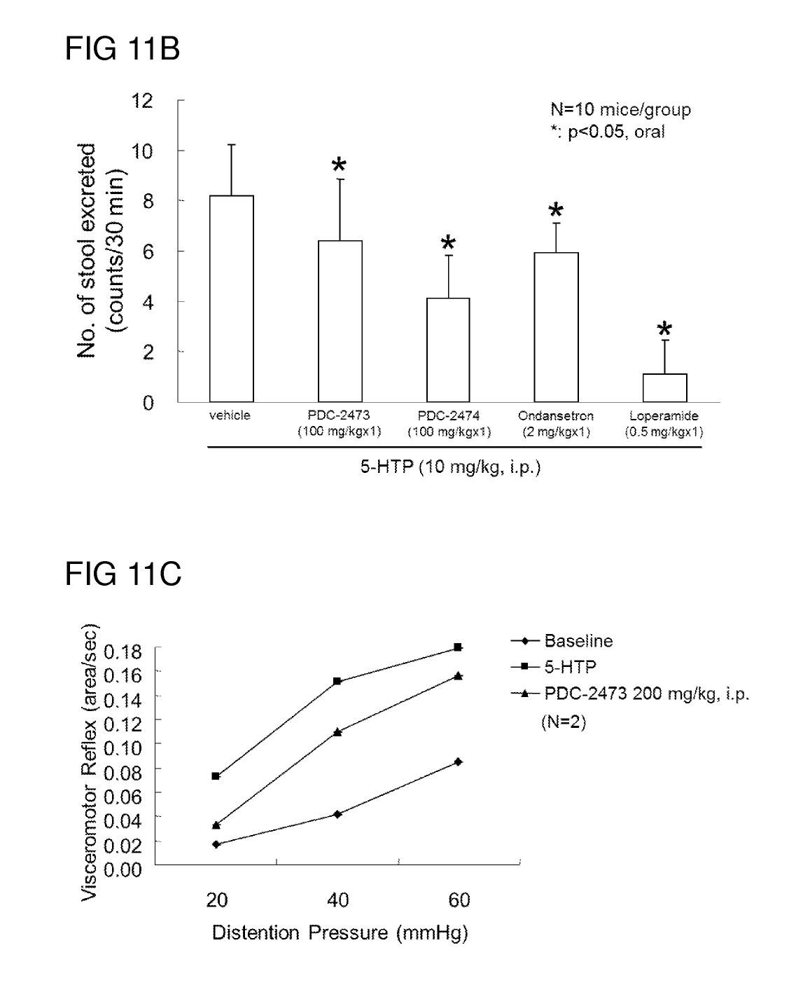

Effects of the Alpinia zerumbet extracts of Example 4 on symptoms associated with IBS are depicted in FIG. 11. Both the water extract (i.e., PDC-2473) and the ethanol extract (i.e., PDC-2474) were capable of lowering the diarrhea score and/or the number of stools excreted in IBS animals (see FIGS. 11A and 11B), as well as the 5-HTP induced visceral hypersensitivity, as compared with that of the control animals (FIG. 11C).

### Example 7

### Compounds of Example 5 Effectively Treating Abnormal Defecation Associated with IBS

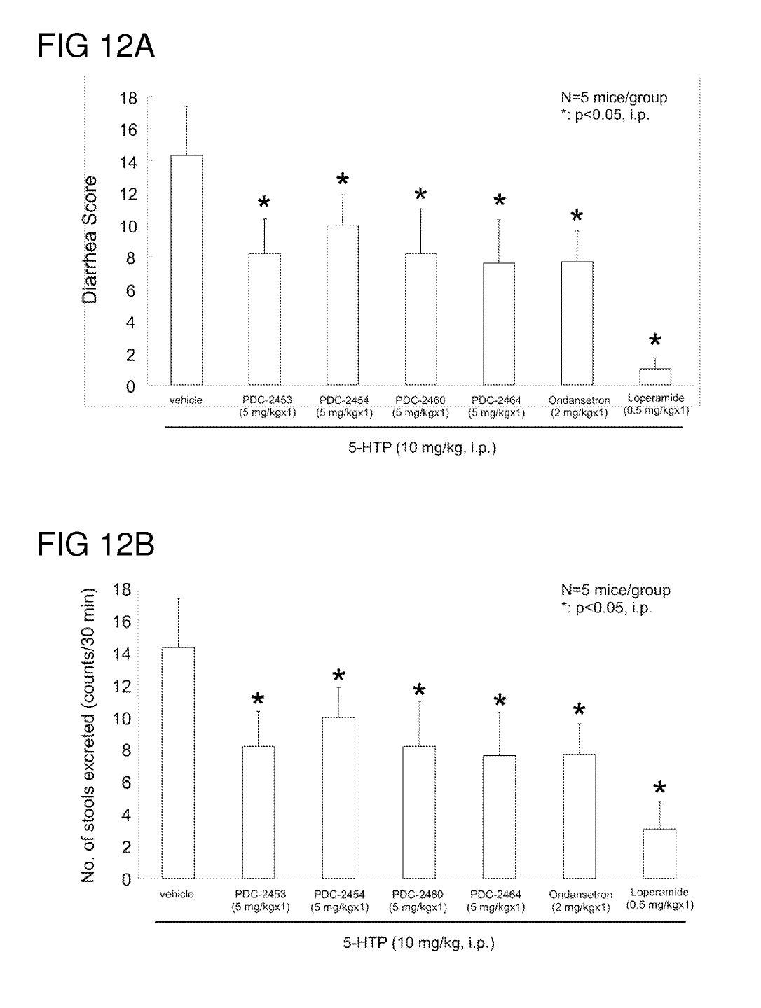

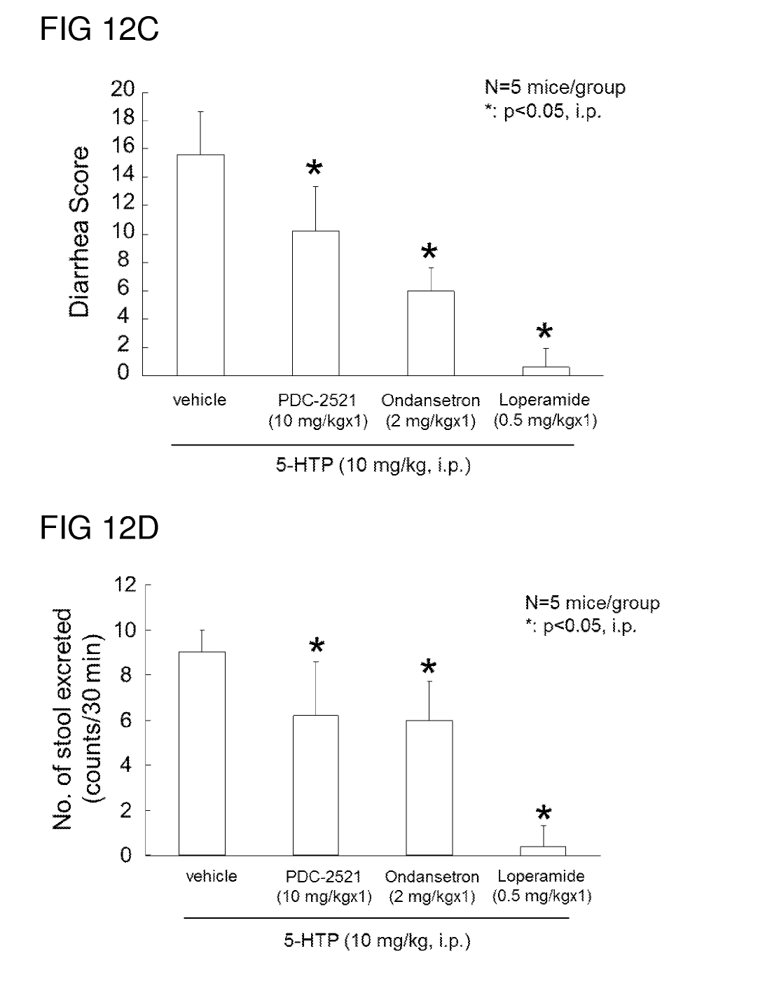

The anti-abnormal defecation effect of the purified compounds isolated from the water extract paste of Alpinia oxyphylla of example 1.1.1.1 (i.e., PDC-2363) was evaluated using the same animal model of Example 6 with similar procedures. Results are illustrated in FIG. 12.

The purified compounds PDC-2453, PDC-2454, PDC-2460, and PDC-2464, are respectively capable of suppressing 5-HTP induced diarrhea (FIG. 12A) and reducing the number of stools excreted (FIG. 12B). Similar anti-abnormal defecation effect was also observed in compound PDC-2521 (FIGS. 12C and 12D). Hence, these compounds are potential candidates for developing therapeutic medicament of IBS.

### Example 8

### Compounds of Example 5 Effectively Treating Abdominal Pain Associated with IBS

The anti-abdominal pain effects of the compounds of example 5 were also investigated by studying the behavioral responses of the animals, which were measured by AWR scores and are summarized in Table 3.

TABLE 3

| | | | body arching and |
|---|---|---|---|
| | | | lifting of pelvic |
| Group | Dose/route | lifting abdomen | structures |
| Sham (n = 5) | | 0.10 ± 0 | 0.10 ± 0 |
| Vehicle (n = 6) | — (10 mL/Kg) | 2.57 ± 0.19 | 2.73 ± 0.15 |
| (10% Tween 80) | | | |
| The compound of | 5 mg/kg | 1.60 ± 0.11* | 1.75 ± 0.16* |
| Example 5.3 | i.p. | | |
| (PDC-2464) | | | |
| (n = 6) | | | |
| The compound of | 2.5 mg/kg | 2.27 ± 0.06 | 2.40 ± 0 |
| Example 5.6 | i.p. | | |
| (PDC-2524) | | | |
| (n = 3) | | | |
| The compound of | 5 mg/kg | 1.87 ± 0.06* | 2.10 ± 0* |
| Example 5.6 | i.p. | | |
| (PDC-2524) | | | |
| (n = 3) | | | |
| The compound of | 10 mg/kg | 1.20 | 1.60 |
| Example 5.6 | i.p. | | |
| (PDC-2524) | | | |
| (n = 1) | | | |

*means significant differences between the compound of example 5 and 5-HTP rat groups (p < 0.05).

As indicated in Table 3, AWR scores in rats treated with the compounds of example 5.3 (i.e., PDC-2464) or example 5.8 (i.e., PDC-2524) are significantly lower as compared with those of the control rats. It is evident that the active compounds isolated from Alpinia oxyphylla are effective in reducing the abdominal pain.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An extract of Alpinia spp. for manufacturing a medicament for treating irritable bowel syndrome (IBS) comprising teucrenone or isalpinin; and at least one active compound selected from the group consisting of oxyphyllenodiol A, 6-α-hydroxy-7-epi-α-cyperone, 7-epi-teucrenone, and tectochrysin.

2. The extract of claim 1, wherein the Alpinia spp. is any of Alpinia oxyphylla, Alpinia zerumbet, Alpinia hainanensis or Alpinia galanga.

3. The extract of claim 2, wherein the extract is prepared by a method comprising steps of,
(a) extracting the Alpinia spp. with a first solvent selected from the group consisting of a supercritical fluid (SFC), water, C₁₋₄ alcohol, acetone, ethyl acetate, or n-hexane; and
(b) drying the extract of step (a).

4. The extract of claim 3, wherein the SFC is any of carbon dioxide, water, methane, ethane, propane, ethylene, propylene, methanol, ethanol or acetone.

5. The extract of claim 4, wherein the step (a) further comprises a co-solvent of the SFC that is methanol or ethanol.

6. The extract of claim 3, wherein the first solvent is any of water, C₁₋₄ alcohol, acetone, ethyl acetate, or n-hexane, and the extraction is performed by mixing the Alpinia spp. with the first solvent in a weight ratio from about 1:10 to 1:50.

7. The extract of claim 6, wherein the method further comprises the step of,
(a-1) adding a second solvent that is any of water, C₁₋₄ alcohol, acetone, ethyl acetate, or n-hexane to the extract before proceeding to the step (b).

8. The extract of claim 7, wherein the method further comprises the step of,
(a-2) subjecting the extract of the step (a-1) to column chromatography before the step (b).

9. The extract of claim 8, wherein the column chromatography is performed by, eluting the column in sequence with water, 30% (v/v) ethanol and 95% (v/v) ethanol; and drying the eluates to produce the extract.

10. The extract of claim 3, wherein the method further comprises the steps of,
(c) dissolving the dried product of step (b) in a second solvent that is any of water, C₁₋₄ alcohol, acetone, ethyl acetate, or n-hexane; and
(d) concentrating or drying the product of step (c).

11. The extract of claim 10, wherein the first solvent is water, and the second solvent is 95% (v %) ethanol.

12. The extract of claim 10, wherein the first solvent is 50% (v %) ethanol, and the second solvent is 95% (v %) ethanol.

13. The extract of claim 12, wherein the method further comprises the step of,
(e) subjecting the solution of step (c) to column chromatography by eluting the column in sequence with 20% (v %) ethanol, 95% (v %) ethanol and acetone before the step (d).

14. The extract of claim 11, wherein the method further comprises the step of,
(f) subjecting the product of step (d) to column chromatography by eluting the column with n-hexane, ethyl acetate and 70-80% (v/v) ethanol.

15. The extract of claim 14, wherein the product of step (f) comprises teucrenone.

16. A method for treating a subject having an irritable bowel syndrome (IBS) in need of such treatment comprising administering an effective amount of the extract of Alpinia spp. of claim 1 to the subject to ameliorate the symptoms of abnormal defecation and abdominal pain associated with the IBS.

17. The method of claim 16, wherein the Alpinia spp. is any of Alpinia oxyphylla, Alpinia zerumbet, Alpinia hainanensis or Alpinia galanga.

18. The method of claim of 17, wherein the extract is prepared by the steps of,
(a) extracting Alpinia spp. with a first solvent selected from the group consisting of a supercritical fluid (SFC), water, C₁₋₄ alcohol, acetone, ethyl acetate, or n-hexane; and
(b) drying the extract of step (a).

19. The method of claim 18, wherein the SFC is any of carbon dioxide, water, methane, ethane, propane, ethylene, propylene, methanol, ethanol or acetone.

20. The method of claim 19, further comprising a co-solvent of the SFC that is methanol or ethanol.

21. The method of claim 18, wherein the first solvent is any of water, C₁₋₄ alcohol, acetone, ethyl acetate, or n-hexane, and the extraction is performed by mixing Alpinia spp. with the first solvent in a weight ratio from about 1:10 to 1:50.

22. The method of claim 21, further comprising the step of,
(a-1) adding a second solvent that is any of water, C₁₋₄ alcohol, acetone, ethyl acetate, or n-hexane to the extract before proceeding to the step (b).

23. The method of claim 22, further comprising the step of,
(a-2) subjecting the extract of the step (a-1) to column chromatography before proceeding to the step (b).

24. The method of claim 23, wherein the chromatography is performed by, eluting the column in sequence with water, 30% (v %) ethanol, and 95% (v %) ethanol; and drying the eluates to produce the extract.

25. The method of claim 18, further comprising the steps of,
(c) dissolving the dried product of step (b) in a second solvent that is any of water, C₁₋₄ alcohol, acetone, ethyl acetate, or n-hexane; and
(d) concentrating or drying the product of step (c).

26. The method of claim 25, wherein the first solvent is water, and the second solvent is 95% (v %) ethanol.

27. The method of claim 25, wherein the first solvent is 50% (v %) ethanol, and the second solvent is 95% (v %) ethanol.

28. The method of claim 27, further comprising the step of,
(e) subjecting the solution of step (d) to column chromatography by eluting the column in sequence with 20% (v %) ethanol, 95% (v %) ethanol and acetone before the step (d).

29. The method of claim 26, further comprising the step of,
(f) subjecting the dried product of step (d) to column chromatography by eluting the column with n-hexane, ethyl acetate and 70-80% (v %) ethanol.

30. The method of claim 29, wherein the product of step (f) comprises teucrenone.
